SEQUENCE LISTING (56 sequences)

SEQ ID NO: 1 - protein, 1230 aa, Lactobacillus plantarum
MIKEVLSVKKSRFFKNVEKFNGTFTQLEENSRRWERLYRERWSHDKVVRTTHGVNCTGSCSWNVYVKQGIITWEHQATDYPECGANMPGYEPRGCPRGASFSWYEYSPVRIKYPYIRGKLWQLWTEAKQTHDNPLAAWASIVENPAKTKIYKSARGHGGMVRVHRPEALELISAMLLYTIKTYGPDRLAGFTPIPAMSMISYASGARFMSLLGGEMLSFYDWYADLPPASPQIWGEQTDVPESADWYNSQYIMMWGSNVPLTRTPDAHFMTEARYHGAKIVAVSPDYAENVKFADNWLAPHPGSDAALAQGMTHVILQEFYQNKQTPEFLNYAKQYTDLPFMVILKPSAANSNDYTSGRFMRISDLQGTDTVANAEWKPVVYDRNQAKVVVPNGTIGQRWEAGQQWNLTLKDADGQAIDPQLNITAEQQVTIDFPTFDADGNGVLKRHVPVQTVTFADGSQQLVTTVYELMLAQYGIDQGNHDQLAASGYDDTNSRYTPAWQETVTGVKASLVVQIAREFAQNALDTGGKTMVIMGAGINHWFNSDMTYRSIINNLMLCGCEGVSGGGWAHYVGQEKLRPQEGWGAIAFANDWQAGGARQQNGTSFFYFASDQWKYDELDNEAQKSPVSASKHAYEHAADYNQLAIRLGWLPSYPQFDRSSLAFAKEYQSTDINEISQHVVDELKSGQLHFAAENPDANQNQPKGLFIWRSNLFTSSGKGQEYFMKHLLGAENGLLAKSNSRCQPEEMVWNDDTVTGKLDLVIDMDFRMVATPMYSDVVLPAATWYEKADLSSTDMHPFIHPFNAAINPMWESKSDWQQFKTLAKTFSTMAKKYYPEPQYDLKTQPLGHDSKGEISQPLGEIRDWKKGEIDAIPGQTMPALSLIKRDYTQVYDKYIALGPNARGKEGGLGFSYDVTTEYDELKDILGVYDRGVNAGCPKLDQDKRVVDAILHISSTSNGHVAKKAWANAETVTGRSLSDISAGRTEEQMSFKSITVQPAEAIPTPIFTSAKHDGDRYSPFTVNIERLVPFRTLTGRQQFYLDHEIFQEYGESLANYKPTLPPQVMAPGDTEIAPADNELTLRYMTPHGKWNIHTMYYDNLEMLTLFRGGPNVWLSPVDADKAGIKDNDWLEIYNRNGVVTARAVVSHRMPAGAMYMYHAQDMEIQEPLSTITGNRGGSHNAPTQIHVKPTQMVGGYGQLSYGFNYYGPIGNQRDLYVNVRKLKKVNWNED

SEQ ID NO: 2 - protein, 519 aa, Lactobacillus plantarum
MKIKAQIGMVLNLDKCIGCHTCSVTCKNTWTNRPGAEYMWFNNVETKPGVGYPKRWEDEDHYKGGWELNSKGKLQLRAGNKVNKIALGKIFYQPDMPELDDYYEPWTYDYQTLFGPEKAHQPVARATSQITGLKMDLKTGPNWDDDLAGSPEYFKADPNMEKIETDIKANFEQAFMMYLPRLCEHCLNAPCVASCPSGAMYKRDEDGIVLVDQERCRGWRFCMTGCPYKKVYFNWKTHKAEKCTFCYPRIEEGEPTVCAETCVGRIRYIGAMLYDADRVEEAAETPEEDQLYQAQLDLFLDPNDPDIIEQALADGISEEMLEAAQNSPIYRMAVEEKIAFPLHPEYRTMPMVWYIPPLSPVMNYFEGRDSIKDPEMIFPGIDEMRIPVDYLASLLTGGNVPAIKGALYKLAMMRLYMRAKTGGKDFDAAKLQRVGLTENTATSLYRLLAIAKYEDRFVIPQNHKEQVEDAQSEQGGLGYDECAGCALAPQHGSMFKKAEAGESTNQIYAESFYGGIWRD

SEQ ID NO: 3 - protein, 190 aa, Lactobacillus plantarum
MINFKQLTTMQPALVTLSRLIDYPDEMTFATATRQNIVTGYPATAQKAKLLAAFDELAAQPLLAQQAHYAGLFEMNKRYTLYMSFYKMTDSRERGTVLAKLKMMYEMFGLTTVSSELADFLPLLLEFLAYGHFADDPRQQDIKLAFQVIEDGTYTMLQNAAAELDDPYFRLLQVVRAELRTCVETGVAAS

SEQ ID NO: 4 - protein, 229 aa, Lactobacillus plantarum
MKDFFSVILWVIYPYAMLLSFFVGTFVRLKFYPASVTAVSSEMLEKKKLMIGAITFHVGIILAFFGHILGILIPKAFTDFLGISDEMYHMFGSLMMGTGAGVLALAGMIILTYRRFTNVRVFVTSSWSDLMVNVALLITIILGLASTLSGPLHPAFDYRTTLSVWARSLFYLQPKWWLMATVPWIYKTHVICGLAIFGFFPYTRLIHAIAIPYQYFYRRYIVYRRRPRV

SEQ ID NO: 5 - DNA, 3693 bp, Lactobacillus plantarum
gtgataaaggaggtcttatcggtgaaaaaatcccgtttctttaaaaatgtcgaaaagttcaacggtacgttcactcagctggaagaaaatagccggcgatgggaacgcttgtatcgtgagcgttggtcccatgacaaagtcgttcgtacgacgcacggggtcaattgtacgggttcgtgtagctggaacgtgtacgttaaacaaggcattatcacttgggaacatcaagcaactgattatccagaatgcggcgcaaacatgccgggctacgagccccgggggtgtccacggggggcgagcttttcgtggtatgaatacagcccagtgcggatcaaatatccatatattcgcggaaagctctggcaactgtggactgaggccaagcagacgcatgataatccgttagcagcttgggctagtattgtcgagaacccagctaaaactaaaatttataaatcggcgcggggccatggtggcatggttcgggttcaccgaccagaggcactcgaattgatttcagccatgttactctatacgatcaaaacatatggcccagaccggctggccggctttacgccgattccggccatgtcgatgattagttatgcttcgggtgcccgctttatgtcgctactcggtggtgagatgttgagcttttatgactggtatgcggatttgccaccggcttcaccacaaatttggggtgagcagaccgatgttcctgaatcagcggactggtataacagtcagtatattatgatgtggggctccaacgtgccattgacgcggacaccggatgcgcactttatgactgaagcgcggtatcacggcgctaagattgtcgcggtcagtccggactacgctgaaaatgtcaagtttgcggacaactggttagccccgcatccgggctcagatgccgccttggcacaaggaatgacccacgttattttgcaagagttttatcagaataaacagacgcctgaatttttgaactatgccaaacagtatacggatttaccattcatggtcatcttgaagccgagtgcggctaattcaaatgattatacgtccgggcgtttcatgcggatctctgatttacaaggtaccgacacggtcgccaatgccgaatggaaaccggtcgtctatgatcgcaatcaagctaaggtcgtggttcccaatgggacgatcggtcagcgttgggaagccggtcagcagtggaatttgacactcaaagacgctgatggtcaggcaattgatccgcagttgaatattacggcggaacaacaagtcacgattgactttccaaccttcgacgccgacggaaacggggttctcaaacgacacgtgccagtgcaaacggttacttttgcggatggtagtcaacagttggtgacgaccgtttacgagctgatgttggcccagtatgggattgatcaaggcaaccatgatcaattggctgccagtggttatgatgatactaatagtcgttatacaccagcttggcaagagacggtgacgggtgttaaagctagtctagtcgttcaaattgcccgtgaattcgctcaaaatgcgctggatactggtggcaagacaatggtcattatgggcgccggtatcaatcattggttcaattccgatatgacctatcgttcaattatcaataacttgatgttgtgcggctgtgaaggggtatccggtggtggctgggcccactatgttggtcaagaaaagttgcggccacaagaaggctggggcgccattgcatttgccaatgactggcaagctggcggtgcccgccaacaaaacgggacctcgtttttctacttcgcatccgatcagtggaaatatgatgaattggataatgaagcgcagaagtcacccgttagtgcgtccaagcatgcttatgaacacgcggctgattataaccagttggcaattcgactgggttggctgccatcctatccacaatttgatcgtagttcgttagcctttgctaaggaatatcaatcgacagatatcaatgaaatttcacagcacgtcgtcgatgaactcaagagcggtcagttacactttgctgctgagaatccagacgccaaccagaatcaacccaagggtctctttatttggcgttcgaacctcttcacatcgtctggtaaggggcaagaatactttatgaaacatttgttgggcgctgaaaatggcctgttagcgaagtctaatagccgttgtcaacctgaagaaatggtttggaatgatgatacggtcactggcaaattagatttggtcatcgatatggacttccggatggtggcgacaccgatgtattcagacgtggtcttaccagctgcaacgtggtatgaaaaggctgacttatcctcaactgatatgcatccgtttatccacccgttcaacgcagcaattaatccgatgtgggaatccaagagtgactggcaacaatttaagaccctggcgaagactttctcaacgatggctaaaaagtattatccagaaccgcagtatgatttaaagacgcaaccgctcggccacgattccaagggcgaaattagtcagccactgggtgaaattcgtgattggaagaagggggaaatcgacgcgattcctggacagaccatgcccgctttgtcattaatcaaacgggattacactcaggtttatgacaagtacattgcgttaggaccgaacgcgcgtggtaaggaaggtggcctgggtttcagttatgatgtcacgactgaatacgatgagcttaaggatattttaggggtgtatgatcgcggtgtgaatgctggttgcccaaagttagatcaagataaacgcgttgtcgatgccatcttgcacatctcgagtacgtccaacgggcacgttgccaaaaaggcttgggctaacgcggaaacggtcactggccggtctttgagtgatatcagtgctggtcgcactgaagaacaaatgtccttcaagagtattacggttcaaccggcggaggcgattccaacgccaattttcacctcggcaaagcatgatggtgatcgttactcaccatttaccgtcaatattgaacggttagtgccatttcggactttgactggccggcaacagttctacctggatcatgaaatcttccaggaatacggggaaagtttggccaattataagccaacactgccaccacaagttatggcacctggtgatacggaaatcgcaccggccgataatgaactgacattgcggtacatgacgccacatggtaagtggaatatccacacgatgtattatgacaatcttgaaatgttgacgttattccggggcgggccgaatgtttggctcagtccagttgacgctgataaagccggcatcaaagataacgattggttggaaatctacaaccgtaacggggtcgtgactgcccgggcggtcgtttcccatcggatgccagcgggggccatgtacatgtatcatgcgcaagacatggaaatccaagaaccgttgtcaacgattacgggtaatcggggtggctcgcacaatgcgccaacccaaattcatgtgaaaccaacccaaatggtcggtggttatggtcagttgagttatggtttcaattattatggcccaatcggtaatcaacgggacctgtacgtgaacgtacggaagttaaagaaggtgaactggaatgaagattaa

SEQ ID NO: 6 - DNA, 1560 bp, Lactobacillus plantarum
atgaagattaaagcacaaattgggatggtcttaaaccttgataagtgtattggttgccatacctgttcggtcacttgtaaaaatacttggaccaatcgtcctggggcagaatacatgtggtttaacaatgtcgagaccaaacccggtgtcggctacccgaaacgctgggaagatgaagatcactataaaggtggttgggagttgaacagtaaaggtaaacttcaactccgagcaggtaataaggtcaataagatcgcactcggaaagattttttatcaacctgatatgccagaattggacgactattatgaaccatggacgtatgattaccaaaccttattcggacctgaaaaggcccatcaaccagttgcacgcgcgacgtcgcagattactggattaaagatggatcttaagacgggccccaactgggatgatgatttagcaggatcacctgaatatttcaaagcagacccgaacatggaaaagatagagactgatatcaaagcgaactttgaacaggccttcatgatgtacctcccacgtctatgtgaacattgcttgaacgcaccatgcgtggcgtcatgtccttctggggcgatgtataagcgtgatgaagacggtatcgtcttggttgatcaagaacggtgccgcggctggcgtttttgtatgacgggttgcccttataagaaggtctacttcaactggaaaacccataaggctgaaaaatgcacgttctgttatccacggatcgaagaaggtgaaccgactgtctgtgccgagacttgtgtcggtcggatccggtatatcggcgccatgttatatgacgctgatcgggtcgaagaagcggccgaaacgcctgaggaagaccaattataccaagcacagttagatttgttcttggaccccaatgatccagacatcattgaacaagcgttggcggatggcatctctgaagaaatgttggaagctgctcagaattcgccgatttaccggatggccgtcgaagaaaagatcgcttttccgcttcatcctgaatatcggacgatgcccatggtttggtacatcccaccattgtcaccagtcatgaattattttgagggccgcgattcgatcaaagatcctgagatgattttcccaggaatcgatgagatgcgcattccagttgattatttggctagtttacttacgggtggcaacgttccggcaatcaagggtgccctctacaagttagccatgatgcggctgtacatgcgggctaagaccggtggcaaggacttcgacgcggctaaattacaacgcgttggcttgactgagaacacggcgacatcactttatcgcttgctggcgattgccaagtacgaagaccgttttgtaattccacaaaatcacaaagaacaagtggaagatgcccagagtgaacagggcggcttaggttatgacgaatgtgctggctgcgcgttagcaccacagcatggcagtatgttcaagaaagccgaagctggtgaatcgaccaatcaaatttacgcagaaagtttctacggggggatttggcgtgattaa

SEQ ID NO: 7 - DNA, 573 bp, Lactobacillus plantarum
gtgattaattttaaacaattaacaacgatgcaacccgcgctagttactttgtcgcggttgatcgactatcctgatgagatgacgttcgctacggctacccgtcagaatatcgtaacgggctatccggcgactgctcaaaaagccaaattgttggcggcattcgatgaactagccgcccaaccgttattagcgcaacaagcccactatgctggcttatttgagatgaacaagcgctacaccctctatatgagtttttacaagatgacggattcccgggaacgggggaccgttttggcgaagctcaagatgatgtatgagatgtttgggctgacgacggttagtagtgagttagcggacttcctgccgctgttgctggaatttctagcgtatggccactttgcggacgatccgcgacaacaggatatcaagctggcttttcaagtcattgaagatggcacctatacgatgttacaaaatgcagcggccgaattggatgatccatacttccggttgttacaagtcgtacgggcggaattacggacgtgtgtggaaacgggggtcgcggcatcatga

SEQ ID NO: 8 - DNA, 690 bp, Lactobacillus plantarum
atgaaagatttcttttccgtaatactgtgggtgatctacccgtatgccatgttattatcgttcttcgtggggacgtttgtgcggctgaaattttatccggctagcgtcacggcagtttccagtgaaatgctggagaagaaaaaactgatgattggggccatcactttccacgttgggatcattttagccttttttgggcatatcttagggattctgattccgaaagcttttacggactttttgggaatttcagatgagatgtaccatatgttcggttccttaatgatggggactggggccggagttttggcccttgccgggatgattatcctgacgtaccggcggtttaccaatgtacgggtgtttgtcaccagttcttggagcgatttaatggtcaacgtggcattgttgattacgatcattttaggattggcttcaacactatccggtcctttacatccggcgtttgactaccggacaacactctcagtgtgggcgcgctcattgttctatctccaaccaaagtggtggttgatggctacagtgccgtggatttacaagacgcatgttatttgcggcttagcaattttcggctttttcccatacacgcggctgattcacgcgattgcaattccttaccagtacttctatcggcgttacatcgtttatcggcggcggccacgagtttag

SEQ ID NO: 9 - protein, 396 aa, Lactobacillus plantarum
MTVEDAQTSTRWRAYLALGLATVAMIVCFMSWSNFAPLAGELAIKFHLSVSQRTLLLATPVLLGSIMRIPVGILSDRYGGKKVYLILMAFILIPLLMITKVHTYGMLLVAALLVGMAGTSFAVGVSYASVWFPPEKQGLALGIVSMGNMGNAVAALTLPSISTSYGFNMVYYFLMVLTVILAVLFAIFCREMPVDKTKTVTGALAVAKESSTWYLSLFYFLTFGLFVSFTNLTPLFLEDIFRVPQVTAGLYAALFAGLCTVVRPIGGAAADKWRPMQMLQWIFIGITVFAVIITLTFSNQSLFIFGIVGAGLIAGFGNGVIFKMVPYVLQGNTGAVTGFVGALGGLGGFFPPLLVGWIHTWTGSYRLGIVLLALTGLVCWYALWRRFIHGDVHIVK

SEQ ID NO: 10 - DNA, 1191 bp, Lactobacillus plantarum
atgactgttgaagatgctcaaacaagtacgcgttggcgggcctatttagcgttaggcttggcaacggtggcgatgatcgtttgctttatgtcatggtcaaactttgcaccattagcgggagagctcgcaatcaagtttcacttgagtgtgtcgcaacggacgctgctactagcaacgccagtactactcgggtcaatcatgcgaattccagtcggaattttgagtgatcggtatggtggtaaaaaggtttatctgatcctgatggccttcattttgatcccactgttgatgattacgaaggtacacacgtacggtatgttgttagtcgcagcgttactcgttgggatggccggaacgtcttttgctgtcggggtttcgtatgcctcagtctggtttccgcctgaaaagcaaggactagcattaggaatcgtcagcatggggaacatggggaatgcggtcgctgcgttgacgttgccatccatatcgacctcgtatgggtttaacatggtttactatttcctgatggtattaacggtcattttggctgtcctgttcgccatcttttgtcgcgaaatgccggttgataagacgaaaactgtgacgggcgcattggctgtcgcaaaggaaagtagtacttggtacttgtcattattttattttctaacgtttggtctgtttgtctcattcactaatttgacaccattgttcttggaagatattttccgtgtgccacaagtaacagcaggcttatacgctgccttatttgctggcctttgtaccgttgttcgaccaattgggggagcggcggccgataagtggcgaccgatgcagatgttacagtggatctttatcggcattaccgtctttgccgtgattattacgctgacctttagcaaccagtcgctgtttatctttggtattgttggtgccggattgattgccggcttcggtaacggggtcatctttaagatggtgccgtatgtattacaaggaaatactggtgcggtcaccggttttgtcggcgctttgggtggcctgggcggtttcttcccaccactattagtggggtggattcatacttggacgggaagttatcggcttggtatcgtgctcttagccctcacgggattagtttgttggtatgccctttggcgccggtttattcatggtgacgtccacattgttaaataa

SEQ ID NO: 11 - protein, 133 aa, Lactobacillus plantarum
MAMIKLSATPLDVNALYQVLKAPEYGGIVTFVGTVRQWTGPIETQSIDYSAYAEMAISQLNKLAAPIEAKGARVVIVHRIGHLDLMDEAVFVGVAAAHRAEAFEWCQYLIDTLKKEVPIWKKEFDTDKVRWGD

SEQ ID NO: 12 - protein, 81 aa, Lactobacillus plantarum
MELTIKLFAMLAEQIGPTVTVTVSTPATAAMVKPALSQRTPALKAVINNARIAVNQEFIADDRQVLQSTDEIALIPPVSGG

SEQ ID NO: 13 - protein, 332 aa, Lactobacillus plantarum
MEKLYDSYDRLHDYVRLSITDRCNLRCVYCMPKEGLPFFPTDRVLSQDEIVQLIENFAAMGVSKVRITGGEPLLRTDVVEIVRRIKAVDGINDVSITTNGLFLAKLAKPLKEAGLDRLNISLDTFKADRYKKITRGGNIQQVLDGIAVASKLHFKKIKLNIVLIKGQNDDEVLDFLHYTKDHDVNARFIEFMPIGNSLKTWQKEYVGLKNVFDTCKDNGLAYHPIVLRGNGPSDNYQIEGYEGSFGLIHPISSKFCENCNRLRITADGYVKACLYWNEEIDIRSAIGDPVAFRKLIQKALDNKPLNHEMAMSETDRIIDKAPTWRHMSQIGG

SEQ ID NO: 14 - protein, 189 aa, Lactobacillus plantarum
MLGIVLAGGQSKRFGRDKAQVQLPGQPLNNVGLAVTKLQLLCEQVIVSASQQNVANLTEQFQTSSNVIVVTDQVPFERQGPLSGIFAATNYSPELTDYLLLAVDYPLITTTILTTLVTQTDCYATTPTQDHYLVSHVQASQDMVRAHLLLGDLRVSHFIKTTCQGLPVTFPDSQAFTNLNNMEALTNAK

SEQ ID NO: 15 - protein, 163 aa, Lactobacillus plantarum
MTDQLTHFNDQNRAKMVDVTDKAVTHRVATATGQITMHPATLQRIHDGQIKKGDVLAVAQVAGIMAAKQTSSLIPMCHLIPLTGVDIHFTDNGQDTITVDALVKTKHVTGVEIEALLAVQVTLLTIYDMCKAIDRGMLINNIHLVEKDGGKSGHFVYPSTPKS

SEQ ID NO: 16 - protein, 165 aa, Lactobacillus plantarum
MALTFQIIGYKKSGKTLITTELVRLLTDRHLHVSVLKHDAHASTMDTPGTDTAQFSHAGAQEVILQSANGIFCHQTTVQPVPVSRLIALLPTSTDVILLEGFKHAPYPKIALLRSDDHAIDFQQFTNIQVFASLTCHPDATLVGKTAICNWFVQTYFKGAATTND

SEQ ID NO: 17 - protein, 406 aa, Lactobacillus plantarum
MPMLTRRYPISITEAQAKINQVALPTKTETIPVTDANHRVLAETVTAPFAYPHFRRSGVDGFAIRHEDDHDYPHEFKVVGNIPAGSTFHQPLGKDEAVRIMTGADVPSDAGVVVMLEKTRELADNRINIVVPEKHSNITEIGEEYQTADVLIEKDTELNPGGLAGLTALGVQTVTVYRQPRVAVITTGSELMAPGEPVQEGKIYNSNGVQIPYLVRENGGVITNVEQLVDDNALLQASLTKAIAENDIVITDGGVSVGDYDFIGDTARQADELLFNKIKQRPGSVTTAFVQDNTLVMALSGNPGACFTAFYLYVEPLLRRFVHQPSRIKKVQSRLAAPYHKTNGFDRILRATFTEDHGQYATYPNGPDRSGALSNLQTTTCLIKIPHSNRPIELNAEVETWLLPFK

SEQ ID NO: 18 - protein, 155 aa, Lactobacillus plantarum
MSRACILTVSDTRDLNTDKSGKLIAERLQHHGVTVMARHVVIDDIVDIQQQFLTFEQLGPDLIITNGGTGIAQRDVTITALTPLLPTMIPGFGEAFRELSFAEIGTRALASKAEAGFNNRNQLCYCLPGSTNACQTALDRLILPEFEHLLFERHK

SEQ ID NO: 19 - protein, 344 aa, Lactobacillus plantarum
MLNRYDRQERVTVIGHDGQRRINAATILIVGVGALGSYAAEQLVRAGVGHLILVDPDTVSLTNLQRQALFTEADVRDQALKVDAAKNHLQAINHHVEITAYPAALDGDLLQTLTFDLVLDCLDNYGTRILINRAALVERFDYIFASCAGTFGTVMPIRAWQHACLNCVYHNLEALQQTDCDLLGVNTALVPIIAGLQVSLALHYLVAPTSVDFEQLTTIDNWQLSQQTFHVRKNPNCPTCQRTDWDLTTAPITNPVQVLCGTATYQARFTQKPNLAAITDWLLDRQFSIKSYASFISFKWEDRPISIFKNGKVMLYNIPDLDAATATFNRLQLYLKSVLEVPIK

SEQ ID NO: 20 - DNA, 402 bp, Lactobacillus plantarum
atggcgatgatcaaattgtctgcgacgccattggatgtgaatgcgttgtatcaagtattgaaggcaccggaatatggcggcattgtcacgtttgtaggcactgttcgccagtggacgggcccaatcgaaacgcaatcaatcgattattcggcgtatgcggagatggccatcagtcaacttaacaaattagcagcgcccattgaagctaaaggcgcccgcgtggtgatcgtgcaccgaattgggcatctggatctgatggatgaagcggtctttgttggtgtcgcggcagcgcaccgtgctgaggccttcgaatggtgccaatacttaattgatacactgaaaaaggaagtcccgatttggaagaaagaatttgataccgacaaggttcgttggggggattaa

SEQ ID NO: 21 - DNA, 246 bp, Lactobacillus plantarum
atggaactaacgattaagttgtttgcaatgctagcggaacaaatcgggccaacggtcacggtgaccgtatcaacgccggcgaccgcggccatggtcaagccagctttgagccaacgaacgccagcgcttaaagctgtaatcaataatgcgcggatcgcggttaatcaagaattcatcgctgatgaccggcaagttttacaatcgactgatgagattgcactaattccgccggtcagtgggggttaa

SEQ ID NO: 22 - DNA, 999 bp, Lactobacillus plantarum
atggaaaaattatatgacagttatgatcgcttgcatgattacgtgcggttatcgattacggatcgttgtaatttgcggtgtgtatattgtatgccgaaagaggggctaccgtttttcccaacggatcgggtcctgtcacaggatgaaattgtccaattgatcgagaattttgcggccatgggagtgtctaaggtccgaatcactggtggcgaaccactgctacggaccgatgtggtcgaaattgttcggcgtatcaaggcggtcgacgggattaacgatgtttcaatcacaactaatgggctatttttggcgaaattagctaagccactgaaagaagccggattggaccggttgaatattagtctggatacgtttaaagcggatcggtacaaaaaaattacgcggggcggtaatattcaacaggttttagatggaatcgccgtggctagtaagttacatttcaaaaagatcaagttgaacattgttttgatcaaggggcaaaatgatgacgaagtcctagatttcttacattacaccaaggaccacgatgttaatgcgcgcttcattgagtttatgccgattgggaattcattaaagacctggcaaaaagaatacgtgggtttaaaaaacgtctttgatacttgtaaagacaacggcttagcctaccatccaatcgttttgcgtggtaacggaccgtctgataattatcaaattgaaggttatgagggaagtttcggtcttattcatccaatcagttctaagttttgtgaaaactgtaatcgattacgaattactgctgacgggtacgtcaaagcatgcttgtattggaacgaagaaatcgatatccggtcggccattggtgatccggtggcattccgcaaacttattcaaaaggcgttagataataagccgctcaatcatgaaatggccatgtccgaaactgaccgcatcattgataaagccccaacgtggcggcatatgagtcagattggaggataa

SEQ ID NO: 23 - DNA, 570 bp, Lactobacillus plantarum
atgttgggaatcgtcctcgctggcgggcagtcgaagcgttttggccgcgacaaagcccaagtccagttacccggccagccgctcaataacgttggtctcgccgtcaccaagttacaactgctatgtgagcaagtcattgtcagtgctagtcagcaaaacgtggctaacttgaccgagcagttccaaaccagttcaaacgttatcgtcgtgaccgatcaagttccttttgagcgccaaggtcccctcagcggtatttttgcagccactaattattcaccagaactgactgattacttgttattggcagttgattatccattaatcactaccaccattttaacgaccttggtcacccaaacagattgttacgcaacgacaccaacacaggatcattacttggtaagtcatgtgcaagccagtcaggacatggtacgcgctcatttattgctaggcgatttacgagttagccactttatcaagacgacatgtcagggactgccggtgacttttccagatagccaagcatttactaatctaaacaatatggaggcacttaccaatgctaagtaa

SEQ ID NO: 24 - DNA, 492 bp, Lactobacillus plantarum
atgactgatcaacttactcactttaacgatcaaaaccgggccaaaatggtcgacgtaaccgacaaagccgtcacccaccgtgtggccacggccactggtcagatcaccatgcacccggccaccctgcaacgcattcacgatggtcaaatcaaaaaaggcgacgtgttggccgtcgctcaagtcgctggtatcatggcggcaaaacaaacgagcagtttaattccaatgtgtcacctgattccactgaccggtgttgatattcattttactgacaacggtcaagatacgatcaccgtcgacgcactcgtcaaaactaagcatgtcaccggtgttgaaatcgaggccttacttgctgttcaagtgaccttgctcacgatttatgatatgtgcaaagctatcgaccgcggcatgctgatcaacaacattcacctcgtagaaaaagacggcggcaaaagtggtcactttgtctatcctagtaccccgaaatcttga

SEQ ID NO: 25 - DNA, 498 bp, Lactobacillus plantarum
atggctcttacctttcaaataattggctacaaaaagagtggcaagacactcatcacgactgagttggtcagactgctgactgatcgccacttacacgtgagcgtcctcaaacacgacgctcacgcaagtacgatggatacgcccggtaccgacacggcacaattcagccacgccggtgctcaggaggtcatcctgcagtccgccaatgggattttttgccatcaaacaaccgtacagcctgtccctgtcagcaggctgattgcactgttaccgacgagtacggacgtgatcttgttggagggttttaaacacgcaccctatccgaaaatagcgctgttacgatccgacgatcacgcgatcgactttcaacaatttacaaatatccaagtctttgccagtctgacttgccatcctgacgcaacgttggtcgggaagacggcaatttgcaactggtttgtacaaacttactttaaaggagctgcgacaactaatgactga

SEQ ID NO: 26 - DNA, 1221 bp, Lactobacillus plantarum
ttgcctatgttaactagaagatatcccatctcaattaccgaagcccaggctaaaatcaaccaagttgcgctaccaacgaaaacggaaacaatccccgtcaccgacgcgaatcaccgggttctagccgaaacggtcaccgcaccattcgcgtacccccactttcgacgctcaggagttgatgggttcgctattcgtcacgaagacgaccatgactaccctcacgaattcaaggtcgtcggtaatatcccagctggatcaacttttcatcaaccccttggcaaagatgaagccgtacggatcatgacgggtgcggacgttccaagcgatgctggcgtcgtcgtgatgctcgaaaagacacgcgaactggctgacaaccgcatcaacatcgtggttcccgaaaagcattctaacatcactgaaattggtgaagaatatcaaactgccgatgttttgattgaaaaggacaccgaacttaatccaggcggcctagccggtttgaccgcactaggcgtccagacagtcaccgtctaccggcagcctcgcgtcgccgtcatcacgactggtagcgaactgatggccccgggtgaacctgtccaagaaggcaaaatttataatagtaacggcgtgcagatcccgtacctcgtccgcgaaaatggtggcgtgatcaccaacgttgaacaattagttgacgacaacgcgctattgcaagcgtccctaactaaagcgattgctgaaaatgatatcgtcattactgacggcggtgtctcagttggtgactacgattttattggcgatactgcgcggcaagccgatgagctcttattcaataaaatcaagcaacggccgggtagtgtcactacggcgtttgttcaggataacacgctcgtcatggccttgtcaggtaatccgggcgcttgctttacggctttctacctatacgttgagccccttttacgccgattcgtccatcagccgagtcgcatcaaaaaggtccagtcacgtttggcagcgccttaccataagaccaacggtttcgaccggatccttcgagccacatttacagaagatcacgggcagtacgcgacgtacccgaatggtcccgaccgctcaggtgccttaagtaatttacaaacaaccacgtgtttgattaaaattccgcacagtaatcgtccgattgaactcaatgccgaggtggaaacatggctcttacctttcaaataa

SEQ ID NO: 27 - DNA, 468 bp, Lactobacillus plantarum
atgagtcgtgcttgtattttaacagtcagtgatacgcgtgatctgaacactgataaatccggaaaactaattgccgaacggctacaacatcacggggtcaccgtaatggcgcgacacgtcgttatcgatgatatcgttgatattcagcaacagtttctaaccttcgaacaactggggccagacctgattatcaccaacggcggcaccggcattgcgcaacgcgatgtgaccattacagcactgacgcccttattgccaaccatgattcctggttttggggaagccttccgtgaactatcttttgcggaaatcggcacccgcgccctggcatccaaggctgaagcgggctttaataatcgtaaccagctctgttactgcttaccaggctcaaccaacgcctgccaaacggcccttgaccgtctgatcttaccagaatttgaacacttactatttgaacgtcataaataa

SEQ ID NO: 28 - DNA, 1035 bp, Lactobacillus plantarum
atgcttaatcgttacgaccgccaagaacgcgtcacggtgattggtcacgacggtcaacgccggatcaacgctgccacgattttaatcgtcggcgtcggcgcgcttggcagttatgctgccgaacagctcgttcgtgccggcgtgggccacttgattctcgtcgatccagatacggtgtcactgaccaacttacaacgccaagctttgttcaccgaagcagacgttcgcgatcaggccttgaaggtcgatgcagctaaaaatcacttacaggccatcaatcaccatgtcgagattacggcctaccccgccgccttagatggcgacctgttgcaaaccttaacgttcgacttagtcttggattgtttggataactatggcactcggattctgatcaaccgtgcggcactcgttgaacgcttcgactatatcttcgctagttgtgccggaacatttgggacagtgatgcccatccgtgcctggcagcacgcctgcttaaactgcgtctaccacaaccttgaagcgctacaacaaacggactgcgacttactcggcgtcaataccgccctcgtcccgattatagctggcttacaagtctcactagccctccattacttggtcgcacccactagcgttgactttgaacaattgaccacgatcgataattggcagctatcgcagcaaacttttcacgtgcgtaagaatcccaactgtccaacgtgccaacgcaccgactgggacttgaccacagcaccaataactaatcccgttcaggtactctgtggcaccgcgacttatcaggcccggttcactcagaaaccgaacttagcagctattactgactggctgcttgaccgccagttcagtattaaaagttacgccagctttattagtttcaagtgggaagatcgaccaatcagtatcttcaagaatggcaaagtcatgttgtataacattcccgatctggatgcggccaccgcaacatttaaccgcttacaattatatctaaaatccgtattggaggtccctatcaaatga

SEQ ID NO: 29 - DNA, 21 bp, Artificial Sequence
  Description of Artificial Sequence Synthetic primer
ccagtcagtaatagctgctaa

SEQ ID NO: 30 - DNA, 21 bp, Artificial Sequence
  Description of Artificial Sequence Synthetic primer
cgataagacctcctttatcac

SEQ ID NO: 31 - DNA, 21 bp, Artificial Sequence
  Description of Artificial Sequence Synthetic primer
cggaagttaaagaaggtgaac

SEQ ID NO: 32 - DNA, 20 bp, Artificial Sequence
  Description of Artificial Sequence Synthetic primer
cgaattctgagcagcttcca

SEQ ID NO: 33 - DNA, 24 bp, Artificial Sequence
  Description of Artificial Sequence Synthetic primer
gcaaaatgatgacgaagtcctaga

SEQ ID NO: 34 - DNA, 26 bp, Artificial Sequence
  Description of Artificial Sequence Synthetic primer
tattctttttgccaggtctttaatga

SEQ ID NO: 35 - DNA, 20 bp, Artificial Sequence
  Description of Artificial Sequence Synthetic primer
gtcgtcgtgatgctcgaaaa

SEQ ID NO: 36 - DNA, 20 bp, Artificial Sequence
  Description of Artificial Sequence Synthetic primer
tcgggaaccacgatgttgat

SEQ ID NO: 37 - DNA, 21 bp, Artificial Sequence
  Description of Artificial Sequence Synthetic primer
gtttgcggacaactggttagc

SEQ ID NO: 38 - DNA, 23 bp, Artificial Sequence
  Description of Artificial Sequence Synthetic primer
tcttgcaaaataacgtgggtcat

SEQ ID NO: 39 - DNA, 22 bp, Artificial Sequence
  Description of Artificial Sequence Synthetic primer
gccacaagtaacagcaggctta

SEQ ID NO: 40 - DNA, 18 bp, Artificial Sequence
  Description of Artificial Sequence Synthetic primer
cccccaattggtcgaaca

SEQ ID NO: 41 - DNA, 20 bp, Artificial Sequence
  Description of Artificial Sequence Synthetic primer
cccaaagcggtaaggttgtt

SEQ ID NO: 42 - DNA, 20 bp, Artificial Sequence
  Description of Artificial Sequence Synthetic primer
cttcacgctggggtcaactt

SEQ ID NO: 43 - DNA, 22 bp, Artificial Sequence
  Description of Artificial Sequence Synthetic primer
ggaattgatgaagccctagcag

SEQ ID NO: 44 - DNA, 20 bp, Artificial Sequence
  Description of Artificial Sequence Synthetic primer
gaatcccacgaccgttatca

SEQ ID NO: 45 - DNA, 20 bp, Artificial Sequence
  Description of Artificial Sequence Synthetic primer
tgatcctcgttccgttgatg

SEQ ID NO: 46 - DNA, 22 bp, Artificial Sequence
  Description of Artificial Sequence Synthetic primer
ccgatggttgcagttgagtaag

SEQ ID NO: 47 - DNA, 20 bp, Artificial Sequence
  Description of Artificial Sequence Synthetic primer
gtggcgacggttcttaccat

SEQ ID NO: 48 - DNA, 20 bp, Artificial Sequence
  Description of Artificial Sequence Synthetic primer
ccctggaagaccaatcgtgt

SEQ ID NO: 49 - DNA, 21 bp, Artificial Sequence
  Description of Artificial Sequence Synthetic primer
ggcagaacagatcaaggaagg

SEQ ID NO: 50 - DNA, 20 bp, Artificial Sequence
  Description of Artificial Sequence Synthetic primer
tatccacttcggcacgctta

SEQ ID NO: 51 - DNA, 23 bp, Artificial Sequence
  Description of Artificial Sequence Synthetic primer
caccgtacccgtagaagttatgc

SEQ ID NO: 52 - DNA, 22 bp, Artificial Sequence
  Description of Artificial Sequence Synthetic primer
ggagaccttgatccaagaacca

SEQ ID NO: 53 - DNA, 20 bp, Artificial Sequence
  Description of Artificial Sequence Synthetic primer
cccatgatggtgcttcacaa

SEQ ID NO: 54 - DNA, 20 bp, Artificial Sequence
  Description of Artificial Sequence Synthetic primer
tcgtggcagcagaggtaatg

SEQ ID NO: 55 - DNA, 20 bp, Artificial Sequence
  Description of Artificial Sequence Synthetic primer
tgatcctggctcaggacgaa

SEQ ID NO: 56 - DNA, 21 bp, Artificial Sequence
  Description of Artificial Sequence Synthetic primer
tgcaagcaccaatcaatacca

(19) United States
(12) Patent Application Publication
(10) Pub. No.: US 2011/0081699 A1
(43) Pub. Date: Apr. 7, 2011

(54) NITRATE REDUCTION BY A PROBIOTIC IN THE PRESENCE OF A HEME

(76) Inventors: Jeroen Hugenholtz, Ede (NL); Brooijmans Rob, Delft (NL); Smid Johannes Elit, Wageningen (NL)

(21) Appl. No.: 12/668,084

(22) PCT Filed: Jul. 11, 2008
(86) PCT No.: PCT/NL2008/050472

§ 371 Date: Apr. 14, 2010

Related U.S. Application Data
(60) Provisional application No. 61/124,817, filed on Jul. 12, 2007.

(30) Foreign Application Priority Data
Jul. 12, 2007 (EP) 07112328.5

(51) Int. Cl. C12P 3/00

(52) U.S. Cl. 435/168

(57) ABSTRACT

The invention relates to a method for reducing nitrate into nitrite wherein a probiotic and/or starter bacteriumis cultivated under anaerobic conditions in the presence of a nitrate, a heme and optionally a vitamin K.

### FIELD OF THE INVENTION

The invention relates to a method for reducing nitrate into nitrite wherein a probiotic and/or a starter bacterium is cultivated under anaerobic conditions in the presence of a nitrate, a heme and optionally a vitamin K.

### BACKGROUND OF THE INVENTION

Lactobacillus plantarum is a versatile species that is used in a variety of economically important dairy, meat, and many vegetable or plant fermentations and found as an inhabitant of the human gastrointestinal (GI) tract. Additionally there is experimental evidence that Lactobacillus species can persist in the gut for >6 days (1, 21). The ability of some Lactobacillus species to reduce nitrate was observed as early as 1955 however since this time little additional research was carried out on this topic. In contrast, the ability of Lactobacillus species to reduce nitrite has been given far more attention (7, 18, 25). Nitrite, the product of nitrate reduction, is a toxic compound (14). Nitrate is a natural compound in green plants and drinking water, additionally it is used as a curing salt in meat fermentations (8, 12, 13). In human consumption habits therefore, the combined intake of nitrate and Lactobacillus species, via fermented food products is quite common.

In order to investigate what conditions give rise to significant nitrate reduction (e.g. production of nitrite) we have used the well-characterized, and fully-sequenced strain Lactobacillus plantarum WCFS1 (10). This strain possesses a full complement of genes necessary to synthesize the protein subunits of the nitrate-reductase complex (narGHJI), the battery of genes to synthesize the molybdopterin co-factor and the nitrite extrusion protein (nary). Interestingly, to this day, there is no evidence that Lactobacillus plantarum WCFS 1 can reduce nitrate to nitrite, giving rise to the assumption that these genes were pseudo-genes.

Whether significant amounts of nitrite might be produced from nitrate in the gut by Lactobacillus species depends on the type of nitrate-reductase present in Lactobacillus species and its dependency on co-factors/environmental conditions. It is an important issue to investigate this, and the results can have major impact on what can be perceived as healthy (or non-healthy) food-combinations. Furthermore, understanding whether and how nitrate reduction pathway is functional in probiotic could open the way to new attractive probiotic cultivation methods on nitrate as main nitrogen source for among other optimal biomass production.

### DESCRIPTION OF THE INVENTION

The present invention is based on the understanding of the nitrate reduction pathway in a probiotic and/or a starter bacterium and especially in lactic acid bacteria.

In a first aspect, the invention relates to a method for reducing nitrate into nitrite wherein a probiotic and/or a starter bacterium is cultivated under anaerobic conditions in the presence of a nitrate, a heme and optionally a vitamin K.

In the context of this invention, probiotic and probiotic bacterium have the same meaning.

In a first preferred embodiment, no other microorganisms are added. Preferably, no other microorganims are added that are known to be able to reduce nitrate into nitrite, such as Bacillus, Pseudomonas, Paracoccus and Escherichia coli.

In the context of the invention, a probiotic is a bacterium which has a beneficial healthy effect when ingested by a subject and a starter bacterium is part of a (starter) culture that is used to inoculate and thus control the acidification process in specific food fermentations. Preferred probiotic bacteria belong to a genus selected from the list consisting of: Lactobacillus, Lactococcus, Leuconostoc, Carnobacterium, Streptococcus, Bifidobacterium, Bacteroides, Eubacterium, Clostridium, Fusobacterium, Propionibacterium, Enterococcus, Staphylococcus, Peptostreptococcus, and Escherichia. A preferred probiotic and/or starter bacterium is a lactic acid bacteria or a Bifidobacteria. Preferred lactic acid bacteria belong to a genus selected from the following list: Lactobacillus, Leuconostoc, Pediococcus, Lactococcus, and Streptococcus. A further preferred probiotic or starter bacterium is a bacterium that is a Lactobacillus or Bifidobacterium species selected from the group consisting of L. reuteri, L. fermentum, L. acidophilus, L. crispatus, L. gasseri, L. johnsonii, L. plantarum, L. casei, L. paracasei, L. murinus, L. jensenii, L. salivarius, L. minutis, L. brevis, L. gallinarum, L. amylovorus, B. bifidum, B. longum, B. infantis, B. breve, B. adolescente, B. animalis, B. gallinarum, B. magnum, and B. thermophilum.

Accordingly in a preferred embodiment, the probiotic and/or starter bacterium is a Lactobacillus plantarum strain.

Accordingly in another preferred embodiment, the probiotic and/or starter bacterium has not been genetically modified. Probiotic and/or starter bacteria are expected to have the narGHJI operon, showing homology to the E. coli genes. These sequences may also be obtained via http://www.kegg.com/kegg-bin/show_genomemap?ORG=1p1&ACCESSION. Preferred amino acid sequences of the narG (nitrate reductase, alpha chain, EC: 1.7.99.4, 1p_{—}1497), narH (nitrate reductase, beta chain, EC: 1.7.99.4, 1p_{—}1498), narJ (nitrate reductase, delta chain, EC: 1.7.99.4, 1p_{—}1499) and narI (nitrate reductase, gamma chain, EC: 1.7.99.4, 1p_{—}1500) proteins from Lactobacillus plantarum WCFS1 are given in SEQ ID NO:1, 2, 3, and 4 respectively. Preferred corresponding coding sequences are given in SEQ ID NO: 5, 6, 7, and 8 respectively. Surprisingly, we demonstrate that if one uses optimal conditions, this operon is functional and may reduce nitrate into nitrite. As demonstrated in the example, this operon is responsible of active reduction of nitrate into nitrite in Lactobacillus plantarum in the presence of a heme and optionally a vitamin K source. Subsequently, nitrite is reduced into ammonia. These genes appear in an island-like structure in the genome, and are co-conserved among other Lactobacillus species. The narGHJI of Lactobacillus plantarum WCFS 1 belongs to the class of heme-dependent dissimilatory nitrate-reductases (10). E. coli possesses three distinct enzymes to accomplish the reduction of nitrate encoded by the napFDAGHBC, narGHJI and narZYWV operons. The best studied is the major dissimilatory nitrate-reductase encoded by the narGHJI operon. This enzyme complex is located in the cytoplasmic membrane, and couples nitrate reduction to formation of proton motive force (22, 26). The narGHJI operon will be called the operon hereafter. The functionality of the operon (functionality test) in a given probiotic and/or starter bacterium may be assessed in vitro by adding a heme source (preferably 2.5 μg/ml) and optionally a vitamin K source (preferably vitamin K2, more preferably 10 μg/ml) in the presence of a nitrate (preferably NaNO₃ 700 mg/L) under anaerobic conditions (N₂-atmosphere) at 37° C. In a preferred embodiment, if after at least two days, there is no detectable decrease (or utilization) of nitrate concentration, the probiotic and/or starter bacterium tested would be said to be non-functional for the method of the invention. More preferably, if after at least one day, there is no detectable decrease (or utilization) of nitrate concentration, the probiotic and/or starter bacterium is said non-functional for the method of the invention. Nitrate is preferably assessed using a colorimetric method (Roche Diagnostics GmbH). Ammonia is preferably assessed using an UV-method (Boehringer Mannheim/R-Biopharm).

If a probiotic and/or starter bacterium is found non-functional, not capable of using nitrate in above functionality test, one may either decide to look for another probiotic and/or starter bacterium or to genetically modify this probiotic and/or starter bacterium to render it functional according to this test by conferring it the ability to utilize nitrate to reduce it into nitrite. In this case, at least one nucleic acid sequence or gene present on the operon (narG, and/or narH and/or narJ and/or narI as earlier presented herein) or homologous thereof as herein defined is preferably introduced into this non-functional probiotic and/or starter bacterium by techniques known to the skilled person and briefly outlined below.

A nucleic acid molecule is represented by its nucleic acid sequence. A homologous nucleic acid sequence (or homologous gene) is herein defined as being a nucleic acid sequence (or gene) which has at least 50% identity with a first nucleic acid sequence (or gene). Preferably, homologous in this context means, at least 55%, 60, 65, 70, 75, 80, 85, 90, 95, 97, 99, 100% identity. A homologous gene or nucleic acid sequence preferably encodes a polypeptide which has a function which is similar with the one of the polypeptide encoded by the first nucleic acid sequence (or gene) compared with. Percentage of identity was determined by calculating the ratio of the number of identical nucleotides in the sequence divided by the length of the total nucleotides minus the lengths of any gaps. DNA multiple sequence alignment was performed using DNAman version 4.0 using the Optimal Alignment (Full Alignment) program. The minimal length of a relevant DNA sequence showing 50% or higher identity level should be 40 nucleotides or longer. In a preferred embodiment, the identity is assessed comparing the whole SEQ ID NO as identified herein.

A polypeptide is represented by its amino acid sequence. A homologous amino acid sequence is herein defined as being an amino acid sequence which has at least 50% identity with a first amino acid sequence. Preferably, homologous in this context also means, at least 55%, 60, 65, 70, 75, 80, 85, 90, 95, 97, 99, 100% identity. A homologous amino acid sequence preferably has a function which is similar with the one of the first amino acid sequence. Percentage of identity is calculated as the number of identical amino acid residues between aligned sequences divided by the length of the aligned sequences minus the length of all the gaps. Multiple sequence alignment was performed using DNAman 4.0 optimal alignment program using default settings. In a preferred embodiment, the identity is assessed comparing the whole SEQ ID NO as identified herein.

Briefly, a nucleic acid construct may be prepared, each comprising a nucleic acid sequence coding for a polypeptide encoded by a gene of the operon as earlier identified. Optionally, a nucleic acid sequence present in a nucleic acid construct is operably linked to one or more control sequences, which direct the production of a polypeptide in a probiotic and/or starter bacterium.

Operably linked is defined herein as a configuration in which a control sequence is appropriately placed at a position relative to a nucleic acid sequence as earlier defined such that the control sequence directs the production of an encoded polypeptide. Expression will be understood to include any step involved in the production of a polypeptide including, but not limited to transcription, post-transcriptional modification, translation, post-translational modification and secretion.

Nucleic acid construct is defined as a nucleid acid molecule, which is isolated from a naturally occurring gene or which has been modified to contain segments of nucleic acid which are combined or juxtaposed in a manner which would not otherwise exist in nature.

Control sequence is defined herein to include all components, which are necessary or advantageous for the expression of a polypeptide. At a minimum, the control sequences include a promoter and transcriptional and translational stop signals.

The invention also relates to an expression vector comprising a nucleic acid construct as earlier defined. Preferably, an expression vector comprises a nucleic acid sequence as earlier defined, which is operably linked to one or more control sequences, which direct the production of an encoded polypeptide in a probiotic. At a minimum control sequences include a promoter and transcriptional and translational stop signals. An expression vector may be seen as a recombinant expression vector. An expression vector may be any vector (e.g. plasmic, virus), which can be conveniently subjected to recombinant DNA procedures and can bring about the expression of a nucleic acid sequence encoding a polypeptide. Depending on the identity of a probiotic and/or starter bacterium wherein this expression vector will be introduced and on the origin of a nucleic acid sequence of the invention, the skilled person will know how to choose the most suited expression vector and control sequences.

In a further aspect, the present invention relates to a probiotic and/or starter bacterium, which comprises a nucleic acid construct or an expression vector as earlier defined. A transformed probiotic and/or starter bacterium expresses at least one polypeptide encoded by a nucleic acid sequence or gene present in the operon or homologous thereof, which is expected to confer it the ability to reduce nitrate into nitrite. Preferably, a transformed probiotic and/or starter bacterium expresses at least two, at least three, and most preferably each polypeptide encoded by each nucleic acid sequence or gene present in the operon or homologous thereof. Therefore, in a preferred embodiment, a probiotic and/or starter bacterium comprises a nucleic acid construct or expression vector comprising a nucleic acid sequence encoding:
a narG polypeptide, said polypeptide having SEQ ID NO:1 or an homologous thereof, and/or
a narH polypeptide, said polypeptide having SEQ ID NO:2 or an homologous thereof, and/or
a narJ polypeptide, said polypeptide having SEQ ID NO:3 or an homologous thereof, and/or
a narI polypeptide, said polypeptide having SEQ ID NO:4 or an homologous thereof.

In addition, in a more preferred embodiment, a transformed probiotic and/or starter bacterium expresses a nucleic acid sequence or gene needed to synthesize the molybdopterin co-factor or homologous thereof and/or a nitrite extrusion protein or homologous thereof. A preferred amino acid sequence of a nitrite exclusion protein of Lactobacillus plantarum WCFS1 is given as SEQ ID NO:9 (nitrite exclusion protein, narK, 1p_{—}1481). This preferred amino acid sequence is preferably encoded by the corresponding nucleic acid sequence given as SEQ ID NO:10. This amino acid or corresponding nucleic acid sequences are preferably used or homologous thereof. Several nucleic acid sequences or genes are needed in order to synthetize the molybdopterin co-factor. In one preferred embodiment, at least one of the following amino acid sequences or homologous thereof are introduced into a probiotic and/or starter culture:
moaE, molybdopterin biosynthesis protein, E chain (1p_{—}1478): SEQ ID NO:11 and/or
moaD, molybdopterin biosynthesis protein, D chain (1p_{—}1479): SEQ ID NO:12 and/or
moaA, molybdopterin precursor synthase (1p_{—}1480): SEQ ID NO:13 and/or
mobA, molybdopterin-guanine dinucleotide biosynthesis protein MobA, (1p_{—}1491): SEQ ID NO:14 and/or
moaC, molybdopterin precursor synthase MoaC (1p_{—}1492): SEQ ID NO:15 and/or
mobB, molybdopterin-guanine dinucleotide biosynthesis protein MobB (1p_{—}1493): SEQ ID NO:16 and/or
moeA, molybdopterin biosynthesis protein MoeA (1p_{—}1494): SEQ ID NO:17 and/or
moaB, molybdopterin biosynthesis protein MoaB (1p_{—}1495): SEQ ID NO:18 and/or
moeB, molybdopterin biosynthesis protein MoeB (1p_{—}1496): SEQ ID NO:19.

These preferred amino acid sequences are preferably encoded by the corresponding nucleic acid sequences given as SEQ ID NO:20, 21,22,23,24,25,26,27,28. Therefore, in this more preferred embodiment, a probiotic and/or starter bacterium comprises a nucleic acid construct or expression vector comprising a nucleic acid sequence encoding:
a narK polypeptide said polypeptide having SEQ ID NO:9 or an homologous thereof, and/or
a moaE polypeptide, said polypeptide having SEQ ID NO:11 or an homologous thereof, and/or
a moaD polypeptide, said polypeptide having SEQ ID NO:12 or an homologous thereof and/or
a moaA polypeptide, said polypeptide having SEQ ID NO:13 or an homologous thereof and/or
a mobA polypeptide, said polypeptide having SEQ ID NO:14 or an homologous thereof and/or
a moaC polypeptide, said polypeptide having SEQ ID NO:15 or an homologous thereof and/or
a mobB polypeptide, said polypeptide having SEQ ID NO:16 or an homologous thereof and/or
a moeA polypeptide, said polypeptide having SEQ ID NO:17 or an homologous thereof and/or
a moaB polypeptide, said polypeptide having SEQ ID NO:18 or an homologous thereof and/or
a moeB polypeptide, said polypeptide having SEQ ID NO:19 or an homologous thereof.

Alternatively, the molybdopterin co-factor is added to the cultivation medium. The choice of a probiotic or starter bacterium will to a large extent depend upon the source of a nucleic acid sequence of the invention. Depending on the identity of a probiotic or starter bacterium, the skilled person would know how to transform it with the construct or vector of the invention. A nucleic acid sequence may be native for the chosen probiotic or starter bacterium. Alternatively, a nucleic acid sequence may be heterologous for the chosen probiotic or starter bacterium. A nucleic acid sequence or polypeptide which has been subjected to any recombinant molecular biology techniques to obtain a variant nucleic acid sequence or polypeptide will be considered as heterologous for the host cell it originated. Methods for transforming bacterial cells are known in the art and are for example described in “Genetics and Biotechnology of Lactic Acid Bacteria”, Gasson and de Vos, eds., Chapman and Hall, 1994. In case a probiotic or starter bacterium is constructed through genetic engineering such that a resulting recombinant host comprises only sequences derived from the same species as the host is, albeit in recombined form, the host is said to be obtained through “self-cloning”. Hosts obtained through self-cloning have the advantage that there application in food (or pharmaceuticals) is more readily accepted by the public and regulatory authorities as compared hosts comprising foreign (i.e. heterologous) nucleic acid sequences. The present invention thus allows the construction of self-cloned L. plantarum and other lactobacillus hosts for food, pharmaceutical or nutraceutical applications (see also de Vos, 1999, Int. Dairy J. 9: 3-10) and such self-cloned hosts are one further preferred embodiment of the invention.

Alternatively according to another preferred embodiment, a probiotic or starter bacterium is found functional in above-defined test. One choose to improve the functionality of said probiotic or starter bacterium by overexpressing, i.e. producing more of a (at least one) polypeptide encoded by a (at least one) gene present in the operon, and/or of a molybdopterin co-factor and/or of a nitrite extrusion protein (or homologous thereof all as earlier defined herein) than the parental cell this host cell derives from produces when both cultured and/or assayed under the same conditions. Alternatively or in combination with former preferred embodiment, the functionality of said probiotic or starter bacterium is improved by conferring it a higher ability to reduce nitrate into nitrite than the parental cell this host cell derives from has when both cultured and/or assayed under the same conditions. In both cases, a native probiotic or starter bacterium was already able to reduce nitrate into nitrite (functional probiotic). However, in both cases (by overexpressing at least one polypeptide encoded by at least one gene present in the operon, and/or a molybdopterin co-factor and/or a nitrite extrusion protein (or homologous thereof) or by conferring it a higher ability to reduce nitrate into nitrite), it is expected that the ability of the obtained probiotic or starter bacterium to reduce nitrate would be improved.

“Producing more” is herein defined as producing more of a polypeptide encoded by a gene present in the operon, and/or of a molybdopterin co-factor and/or of a nitrite extrusion protein (or homologous thereof) than what the parental host cell the transformed host cell derives from will produce when both types of cells (parental and transformed cells) are cultured under the same conditions. Preferably, the conditions are anaerobic in the presence of a heme and optionally a vitamin K source as earlier defined herein. Preferably, the host cell of the invention produces at least 3%, 6%, 10% or 15% more of a polypeptide encoded by a gene present in the operon, and/or of a molybdopterin co-factor and/or of a nitrite extrusion protein (or homologous thereof) than the parental host cell the transformed host cell derives from will produce when both types of cells (parental and transformed cells) are cultured under the same conditions. Also hosts which produce at least 20%, 30%, 40%, 50%, 60%, 70%, 80%, 90%, 100% or 150% more of said polypeptide than the parental cell are preferred. According to another preferred embodiment, the production level of a polypeptide encoded by a gene present in the operon, and/or of a molybdopterin co-factor and/or of a nitrite extrusion protein (or homologous thereof) is compared to the production level of the Lactobacillus plantarum strain WCFS1, which is taken as control. The Lactobacillus plantarum strain WCFS1 is a single colony isolate of strain Lactobacillus plantarum NCIMB8826 (National Collection of Industrial and Marine Bacteria, Aberdeen, U.K.).

According to an even more preferred embodiment, when a host cell of the invention is a Lactobacillus plantarum strain, the production level of a polypeptide encoded by a gene present in the operon and/or of a molybdopterin co-factor and/or of a nitrite extrusion protein (or homologous thereof) is compared to the production level of the Lactobacillus plantarum strain WCFS1, which is taken as control.

In an even more preferred embodiment, “Producing more” is herein defined as producing more of each polypeptide encoded by each gene present in the operon, and/or a molybdopterin co-factor and/or a nitrite extrusion protein (or homologous thereof) than what the parental host cell the transformed host cell derives from will produce when both types of cells (parental and transformed cells) are cultured under the same conditions. Preferred conditions are the same as above. Even more preferably, “producing more” means producing at least 3%, 6%, 10%, 15%, 20%, 30%, 40%, 50%, 60%, 70%, 80%, 90%, 100% or 150% more of each polypeptide encoded by each gene present in the operon, and/or a molybdopterin co-factor and/or a nitrite extrusion protein (or homologous thereof) than the parental host cell the transformed host cell derives from will produce when both types of cells (parental and transformed cells) are cultured under the same conditions.

The assessment of the production level of a polypeptide may be performed at the mRNA level by carrying out a Northern Blot or an array analysis and/or at the polypeptide level by carrying out a Western blot. All these methods are well known to the skilled person.

“Exhibiting a higher ability to reduce nitrate into nitrite” is herein defined as exhibiting a higher ability to reduce nitrate into nitrite than the one of the parental host cell the transformed host cell derives from using an assay specific for detecting nitrate reduction. Preferably, the assay is the one, which has been already described herein. Preferably, the host cell of the invention exhibits at least 3%, 6%, 10% or 15% higher ability to utilize nitrate than the parental host cell the transformed host cell derives from will exhibit as assayed using the specific assay as already defined. Also host which exhibits at least 20%, 30%, 40%, 50%, 60%, 70%, 80%, 90%, 100% or 150% more of said activity than the parental cell are preferred. According to another preferred embodiment, the ability to utilize nitrate of a probiotic is compared to the corresponding activity of Lactobacillus plantarum strain WCFS1, which is taken as control. According to a more preferred embodiment, when a probiotic is a Lactobacillus plantarum strain, the ability of the probiotic to reduce nitrate into nitrite is compared to the corresponding ability of the Lactobacillus plantarum strain WCFS1, which is taken as control.

The overexpression may have been achieved by conventional methods known in the art, such as by introducing more copies of a nucleic acid sequence encoding a polypeptide into a probiotic or a starter bacterium, be it on a carrier or in the chromosome, than naturally present. Alternatively, a nucleic acid sequence can be overexpressed by fusing it to highly expressed or strong promoter suitable for high level protein expression in the selected organism, or combination of the two approaches. The skilled person will know which strong promoter is the most appropriate depending on the identity of the chosen probiotic or starter bacterium. Preferably when a probiotic is a Lactobacillus plantarum strain, a strong promoter is the NISIN promoter (Pavan S. et al, Appl Environ Microbiol. 2000 October; 66(10):4427-32.) or the pEPN promoter (see also Rud I, et al, Microbiology. 2006 April; 152 (Pt 4):1011-9 and Sørvig E, et al., Microbiology. 2005 July; 151 (Pt 7):2439-49).

Alternatively or in combination with former preferred embodiments, a probiotic or starter bacterium has been genetically modified to produce a heme and optionally a vitamin K. In this case, there is no need to add these compounds (a heme and optionally a vitamin K). In this case, a nucleic acid sequence encoding a given polypeptide may be introduced into a probiotic or starter bacterium the same way as earlier described.

For example, the total or partial capacity to biosynthesize a heme may be introduced into a probiotic or starter bacterium: one or both B. subtilis heme operons or only some of the genes present on these operons or homologous thereof may be transferred to a probiotic or starter bacterium as described in WO 01/21808. Preferably, B. subtilis hemA, hem L, hemB, hemC, hemD, hemE and/or hemH genes or homologous thereof are introduced into a probiotic or starter bacterium.

Alternatively or in combination with former paragraph, the total or partial capacity to biosynthesize a vitamin K may be introduced into a probiotic or starter bacterium: one or some or all of the genes present on the men operon of B. subtilis or homologous thereof may be transferred to a probiotic or starter bacterium as described in WO 01/21808. Preferably, B. subtilis menF, menD, menB, menE, and/or menC genes or homologous thereof are introduced into a probiotic or starter bacterium. Homologous is given the same meaning as earlier defined herein.

A method of the invention may be carried out using any type of matrix. In a preferred embodiment, a method is carried out in a liquid or in a solid or semi-solid matrix. In another preferred embodiment, a method is carried out in or on a product, preferably in or on a food product.

A product might contain endogenous microorganisms. It is preferred that these endogenous microoganisms are not able to reduce nitrate or are not as functional as a probiotic used as assessed using the assay as defined earlier herein.

Any type of product may be used in the method of the invention. Several types of products are below exemplified: food product, pharmaceutical product. Pharmaceutical product will usually comprise a pharmaceutical carrier. The preferred form depends on the intended mode of administration and (therapeutic) application. The pharmaceutical carrier can be any compatible, nontoxic substance suitable to deliver the probiotic of the invention to the GI-tract of a subject. E.g. sterile water, or inert solids may be used as the carrier usually complemented with pharmaceutically acceptable adjuvants, buffering agents, dispersing agents, and the like. Compositions will either be in liquid, e.g. a stabilized suspension of the host cells, or in solid forms, e.g. a powder of lyophilized host cells. E.g. for oral administration, a probiotic can be administered in solid dosage forms, such as capsules, tablets, and powders, or in liquid dosage forms, such as elixirs, syrups, and suspensions. A probiotic of the invention can be encapsulated in gelatin capsules together with inactive ingredients and powdered carriers, such as e.g. glucose, lactose, sucrose, mannitol, starch, cellulose or cellulose derivatives, magnesium stearate, stearic acid, sodium saccharin, talcum, magnesium carbonate and the like.

A preferred product according to the invention is suitable for consumption by a subject, preferably a human or an animal. Such compositions may be in the form of a food supplement or a food or food composition, which besides a probiotic and/or a starter bacterium also contains a heme, a nitrate and/or optionally a vitamin K and a suitable food base. Preferably, a heme and/or optionally a vitamin K are not present in said food product but are added to the food product together with a probiotic and/or starter bacterium and/or are produced by a probiotic and/or starter bacterium itself. Alternatively, a food product may contain detectable levels of a heme and/or optionally a vitamin K and additional amounts of a heme and/or optionally a vitamin K are added to said food product.

A food or food composition is herein understood to include liquids for human or animal consumption, i.e. a drink or beverage. A food or food composition may be a solid, semi-solid and/or liquid food or food composition, and in particular may be a dairy product, such as a fermented dairy product, including but not limited to a yoghurt, a yoghurt-based drink or buttermilk. Such foods or food compositions may be prepared in a manner known per se, e.g. by adding a probiotic and/or starter bacterium to a suitable food or food base, in a suitable amount. In a further preferred embodiment, a probiotic or host cell is a micro-organism that is used in or for the preparation of a food or food composition, e.g. by fermentation. Examples of such micro-organisms include baker's or brewer's yeast and lactic acid bacteria, such as probiotic lactic acid and/or starter bacteria strains as earlier exemplified herein. In doing so, a host cell or probiotic of the invention may be used in a manner known per se for the preparation of such fermented foods or food compositions, e.g. in a manner known per se for the preparation of fermented dairy products using lactic acid bacteria. In such methods, a probiotic may be used in addition to the micro-organism usually used, and/or may replace one or more or part of the micro-organism usually used. For example, in the preparation of fermented dairy products such as yoghurt or yoghurt-based drinks, a food grade lactic acid bacterium and/or starter bacterium of the invention may be added to or used as part of a starter culture or may be suitably added during such a fermentation.

Preferably, the above product will contain a probiotic and/or starter bacterium in amounts that allow for convenient (oral) administration of the probiotic, e.g. as or in one or more doses per day or per week.

In the context of the invention, “anaerobic” preferably means that a method herein defined is carried out in the absence of oxygen or wherein substantially no oxygen is consumed, preferably less than 5, 2.5 or 1 mmol/L/h, more preferably 0 mmol/L/h is consumed (i.e. oxygen consumption is not detectable), and wherein organic molecules serve as both electron donor and electron acceptors.

A heme or haem is a prosthetic group that consists of an iron atom contained in the center of a large heterocyclic organic ring called a porphyrin. Not all porphyrins contain iron, but a substantial fraction of porphyrin-containing metalloproteins have heme as their prosthetic subunit; these are known as hemoproteins. Several types of hemes are known to the skilled person: heme A, B, C, O, Several types of hemes might be added in a method of the invention. Also a heme precursor such as protoporphyrin IX may be used. Preferably, a mixture of heme a, b and/or c is used. More preferably, Hemin from Sigma cat. No. H5533 is used, which comprises a mixture of heme types. a, b, c. Heme a is present in the cytochrome c oxidase, heme c in cytochrome c and heme b for example in haemoglobin.

A heme is preferably used in an amount which is ranged between 1.25 and 50 μg/ml (final concentration in a matrix), more preferably between 1.50 and 25 μg/ml, even more preferably between 1.75 and 15 μg/ml, even more preferably between 2 and 10 μg/ml, even more preferably between 2.3 and 5 μg/ml and most preferably between 2.5 and 5 μg/ml. Very good results were obtained with about 2.5 μg/ml.

Vitamin K is a group name for a number of related compounds, which have in common a methylated naphthoquinone ring structure, and which vary in the aliphatic side chain attached at the 3-position. In the context of the invention, any related vitamin K compound may be used in the method of the invention. Phylloquinone (also known as vitamin K₁) invariably contains in its side chain four isoprenoid residues, one of which is unsaturated. Vitamin K2 also named menaquinone is also a vitamin K compound. Preferably, vitamin K2 is used. More preferably, vitamin K2(4) or Menaquinone-4 is used.

A vitamin K is preferably used in an amount which is ranged between 5 and 100 μg/ml (final concentration in a matrix), more preferably between 7 and 80 μg/ml, even more preferably between 8 and 40 μg/ml, even more preferably between 9 and 20 μg/ml, and most preferably between 10 and 12 μg/ml. Very good results were obtained with about 10 μg/ml.

Accordingly in a preferred embodiment, a heme is present in an amount which is ranged between 1.25 and 50 μg/ml and optionally a vitamin K between 5 and 100 μg/ml.

A nitrate source may be present in a product. Alternatively, a nitrate may be added at the onset and/or during a method of the invention. Preferably, a final concentration of a nitrate in a matrix (preferably NaNO3) is ranged between 100 and 2000 mg/L at the onset of the method, more preferably, between 200 and 1500 mg/L, even more preferably between 400 and 1000 mg/L, and most preferably between 500 and 900 mg/L. Very good results were obtained with about 700 mg/L. Alternatively, nitrate may be present in the complex medium itself.

A glucose source may be present in a matrix or in a product or in a medium. Preferably, a glucose source comprises between 2 and 20 mM glucose (final concentration in a matrix or product), more preferably between 5 and 10 mM glucose.

Depending on the probiotic or starter bacterium, the type of matrix, the amount of a heme and optionally of a vitamin K used, the method of the invention may extend from one day or more till one month or more. Preferably, a method of the invention extends from two days or more till two weeks or more. Usually at least 30% of the nitrate initially present will be reduced into nitrite. Preferably, at least 40%, 50%, 60%, 70%, 80%, 90% or more of the initially present nitrate has been reduced into nitrite. Usually the formed nitrite is subsequently converted into ammonia. Therefore, usually at least 30% of the nitrate initially present will be reduced into ammonia. Preferably, at least 40%, 50%, 60%, 70%, 80%, 90% or more of the initially present nitrated has been reduced into ammonia. In a preferred embodiment, there is a detectable decrease of the initial nitrate concentration at the end of a method of the invention. More preferably, the nitrate concentration as measured at the end of the method of the invention is decreased of at least 5% by comparison with the initial nitrate concentration. Even more preferably, the decrease is of at least 10%, 20%, 30%, 40%, 50%, 60%, 70%, 80%, 90% or more. Even more preferably, nitrate is not detectable at the end of the method of the invention. The presence of all these compounds is preferably assessed as earlier defined herein.

Advantageously, during a method of the invention, the formation of nitrite is expected to prevent outgrowth of spoilage microorganisms such as clostridia, to increase biomass production of the nitrate reducing organism, and even to confer increased stress resistances as with respiration (Duwat, P., S. et al., (2001), J Bacteriol 183:4509-16 and Rezaiki, L., B. et al, (2004) Mol Microbiol 53:1331-42).

Therefore, a method of the invention may be seen as a preservation method for any type of matrix, preferably for any type of product, more preferably for any type of food or pharmaceutical product. Since nitrite is subsequently converted into ammoniac, the obtained matrix, preferably product is expected to be non-toxic and edible.

Another advantage of applying these probiotic and/or starter bacteria is that the nitrate initially present in a food material or product is considerably reduced or even absent at the time of retail and consumption.

In addition, a probiotic and/or starter bacterium present in the matrix, preferably product is expected to have improved characteristics as further exemplified below.

In another preferred embodiment, a cultivation method of the invention allows to obtain a probiotic or starter culture having improved characteristics as to its biomass production and/or its ability to survive in the human or animal gastrointestinal tract. According to a more preferred embodiment, the probiotic and/or starter bacterium hence resulting from this method has an improved biomass production i.e. produces more biomass than the parental cell this cell derives from when both cultured and/or assayed under the same conditions.

“Improved biomass” is herein defined as producing at least 3%, 6%, 10% or 15% more biomass than the parental host cell the host cell obtained with this method will produce when both types of cells (parental and cell obtained with the method) are cultured under the same conditions. Also a cell obtained with the method which produces at least 20%, 30%, 40%, 50%, 60%, 70%, 80%, 90%, 100% or 150% more biomass than a parental cell is preferred. According to another preferred embodiment, the biomass is compared to the biomass production of the Lactobacillus plantarum strain WCFS1, which is taken as control. According to an even more preferred embodiment, when the host cell of the invention is a Lactobacillus plantarum strain, the biomass production is compared to the biomass production of the Lactobacillus plantarum strain WCFS1, which is taken as control.

The assessment of the biomass production level may be performed by measuring the Optical Density (OD) at 600 nm of the cells and/or counting cells under the microscope after an overnight assay in a defined medium. All these methods are well known to the skilled person.

“Exhibiting an improved ability to survive in the human or animal gastrointestinal tract” is herein defined as exhibiting an improved ability to survive compared to the corresponding ability of the parental host cell using an assay specific for assessing this characteristic (Powelsa P. H. et al., International Journal of Food Microbiology Volume 41, Issue 2, 26 May 1998, Pages 155-167) Preferably, a probiotic or starter bacterium cell obtained in this method exhibits at least 3%, 6%, 10% or 15% higher survival rate in the human or animal gastrointestinal tract than the parental host cell the probiotic or starter bacterium cell obtained in this method will exhibit as assayed using the specific assay as already defined. Also host which exhibits at least 20%, 30%, 40%, 50%, 60%, 70%, 80%, 90%, 100% or 150% more of said activity than the parental cell are preferred. According to another preferred embodiment, the ability to survive is compared to the corresponding activity of Lactobacillus plantarum strain WCFS1, which is taken as control. According to a more preferred embodiment, when the probiotic cell is a Lactobacillus plantarum strain, the ability of the probiotic to survive is compared to the corresponding ability of the Lactobacillus plantarum strain WCFS1, which is taken as control.

In a second aspect, the invention provides an aerobic cultivation method of a probiotic and/or starter bacterium in the presence of a heme, a nitrate and optionally a vitamin K for obtaining a probiotic and/or starter bacterium having improved characteristics as to its ability to survive in the gastrointestinal tract All features of this aspect have already been earlier defined herein.

In the context of this aspect, “aerobically” is opposed to “anaerobically” as earlier defined herein.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a probiotic, a starter, a product or a composition as defined herein may comprise additional component(s) than the ones specifically identified, said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. Each embodiment as described herein may be combined with other embodiment(s) as described herein unless otherwise indicated.

All patent and literature references cited in the present specification are hereby incorporated by reference in their entirety.

The present invention is further described by the following examples, which should not be construed as limiting the scope of the invention.

### DESCRIPTION OF THE FIGURES

FIG. 1. Wild-type cells were grown overnight in nitrate-MRS medium, containing variable amounts of glucose. An optimum concentration of glucose (10 mM) is observed for nitrite production. High glucose levels correlate with high biomass production, however not with high nitrite levels.

FIG. 2. Genes encoding a nitrate-reductase A complex (narGHJI) and a quinol-oxidase bd complex indicate the existence of a branched electron chain in Lactobacillus plantarum WCFS. Since both pathways are activated under different condition (e.g. in the presence of oxygen or nitrate) these reflect alternative (non-competitive) electron transport chains.

FIG. 3. Ct-values compared of wild-type and narGΔ. cells both grown in nitrate-MRS. recA, rpoB, fusA, GroES, gyrB, ldh are selected household genes. Representatives of nitrate-reductase (related) genes all have higher Ct-values in the narGΔ. (lower transcription levels in narGΔ. compared to wild-type cells).

FIG. 4. Biomass production by wild-type and narGΔ after overnight incubation on nitrate-MRS, and wild-type on nitrate-MRS without nitrate (but with +heme and +vitamin K₂).

FIG. 5. Growth of Lb. plantarum WCFS1 on chemically defined medium supplemented with heme, vitamin K2, 20 mM nitrate and 10 mM glucose (nitrate-CDM) followed in time.

### EXAMPLES

### Materials and Methods

### Cultures and Growth Conditions

The principal strains used in this study are Lactobacillus plantarum WCFS1, an isolate from NCIMB8826, and NarGΔ, a derived mutant, lacking a complete narG-gene (10). Lb. plantarum strains were grown on MRSB (Man, Rogosa and Sharpe Broth) (Difco), with(out) citrate and acetate when mentioned, or chemically defined media (CDM, see appendix 1). When NarGΔ was grown the medium was supplemented with 5 μg/ml chloramphenicol. For the induction of nitrate-reductase activity heme (or hemin, Sigma-Aldrich, H5533) was added to a final conc. of 2.5 μg/ml (stock 0.5 mg/ml in 0.05 M NaOH Sigma-Aldrich), vitamin K2 (or menaquinone 4) to a final conc. of 1 μg/ml (stock 2 mg/ml in ethanol Sigma-Aldrich) and NaNO₃ (Sigma-Aldrich) to 700 mg/L. For nitrite-reduction assays, NaNO₂ (Sigma-Aldrich) was added to a final conc. of 500 mg/L. Cultures were grown anaerobic under N₂-atmosphere at 37° C. Escherichia coli were grown aerobically at 37° C. on TYB-medium (Difco) with 10 ug/ml chloramphenicol and/or 10 ug/ml erythromycin, when appropriate.

### Mutant Construction

Molecular cloning techniques were carried out in accordance with standard laboratory procedures (19). To construction the mutant lacking a complete narG (NarGΔ), via a double crossover event, the knock-out plasmid (pNZ5319_NarG_KO) was constructed, based on pNZ5319, containing up- and downstream flanking sequences of the Lb. plantarum WCFS1 narG-gene (Lambert, J. M., R. S. Bongers, and M. Kleerebezem Appl Environ Microbiol. 2007 73(4): 1126-1135,). A 1 kb fragment upstream (forward primer p101: CCAGTCAGTA ATAGCTGCTA A, reverse primer p100: CGATAAGACC TCCTTTATCA C) and downstream of narG (forward primer p102: CGGAAGTTAA AGAAGGTGAA C, reverse primer p103 CGAATTCTGA GCAGCTTCCA) were PCR amplified (for primers sequences see table 1). The flanking fragments were cloned, using Escherichia coli as host strain, blunt-ended in vector pNZ5319 digested with SwaI (upstream fragment) and Ec136II (downstream fragment) to produce the knock-out vector pNZ5319_NarG_KO. The knock-out plasmid was transformed into Lb. plantarum WCFS1 and a chloramphenicol replacement of the narG gene was obtained by a double cross over event by homologous recombination which resulted in the mutant strain NarGΔ.

### Transcriptional Anaylsis

To quantify the differential expression of the different operons involved in producing the nitrate-reductase proteins, molybdopterin-cofactor and nitrite-extrusion enzyme, we performed Q-RT-PCR. Amplification was carried out in 96-well plates in an ABI Prism 7700 from Applied Biosystems using the fluorescent agent SYBR (SYBER green), Green for detection. Reactions were set up using the SYBR Green Master Mix from the same manufacturer following its recommendations. Specificity and product detection were checked after amplification by determining the temperature dependent melting curves. Primers were designed with the Primer Express software package (Applied Biosystems, The Netherlands) to have a Tm between 59 and 61° C. and an amplicon size of 100±20 by (Table 1).

### Determination of Nitrite and Nitrate

Nitrate and nitrite were determined by photometric endpoint determination, using a “Nitrite/nitrate, colorimetric method” kit from Roche Diagnostics GmbH, Mannheim Germany as described (2, 6, 11).

### Determination of Acetic Acid

Acetic acid was determined by a UV-method using the “Acetic acid” kit from Boehringer Mannheim/R-biopharm as described (3-5)

### Determination of L-Lactate

L-lactate acid was determined by a UV-method using the “D-Lactic acid/L-Lactic acid” kit from Boehringer Mannheim/R-biopharm as described (9, 16, 17)

### Results

### Effect of Medium Composition on Nitrite Production

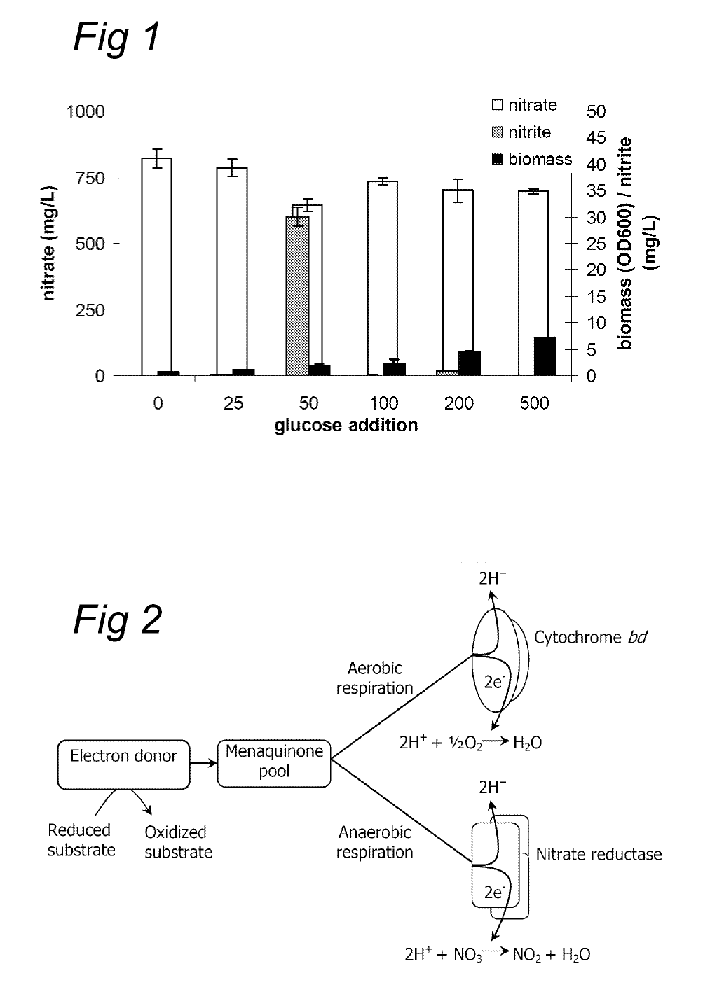

Lb. plantarum WCFS1, when cultivated overnight in MRS supplemented with nitrate (nitrate-MRS) produces no detectable nitrite. This type of nitrate-reductase is a heme-dependent protein complex, however adding heme to nitrate-MRS did not stimulate the production of nitrite. In E. coli this nitrate-reductase complex operates in an electron transport chain context requiring mena(quinones) (Bonnefoy V., et. al., 1994 Antonie Van Leeuwenhoek 66:47-56). The genome of Lb. plantarum WCFS reveals no capability to produce (mena)quinones. Adding a source of menaquinone (vitamin K2) to nitrate-MRS did produce a small, but measurable, level of nitrite after an overnight incubation. Addition both heme and vitamin K2 did not lead to a further increase in nitrite production. That vitamin-K2 alone in MRS-medium induces small amounts of nitrite being formed, is not surprising since the meat-extract component of MRS contains trace amounts of heme. When cells were grown in batch in nitrate-MRS in which glucose levels were titered, high levels of nitrite were formed at lower glucose concentrations, the highest levels at 10 mM glucose (FIG. 1). Thus surprisingly high biomass formation does not correlate with formation of high nitrite levels. The absence of nitrite formation at high glucose levels can be caused by catabolite-repression of the nitrate-reductase complex and/or inhibition of the activity by low pH. Using 10 mM glucose in chemically defined medium we demonstrated that both heme and vitamin-2K are required for nitrite production (tab 2).

### Nitrate-Reductase Genes in Lactic Acid Bacteria

The Lactobacillus plantarum WCFS narGHJI operon shows high levels of homology to the heme-dependent E. coli nitrate-reductase genes (tab 3).

The presence of these genes in Lactic Acid bacteria seems to be rare. Of the 50 partially or fully sequenced strains lactic acid bacteria, among which 12 Lactobacillus sp., only Lactobacillus reuteri 100-23 and Lactobacillus plantarum WCFS 1 have these genes annotated (http://www.nebi.nlm.nih.gov/blast/).

In two separate papers, the genotypic diversity of in total 24 Lactobacillus plantarum strains was studied by genotyping (15) (Tseneva et.al. submitted for publication). These reveal a high occurrence of nitrate reductase genes (narGHJI), genes to synthesize the molybdopterin co-factor and the nitrite extrusion protein (nark). In Lactobacillus plantarum sp, the full complement of these genes is present in roughly half of the strains tested (data not shown) Lactobacillus plantarum WCFS 1 these genes lie in close proximity to each other, forming an island (10). The co-occurrence or/co-absence of this entire gene-set in the other strains suggest a similar genetic topography. Of the 26 genes present within this conserved genetic nitrate-reductase island 14 have clear function in producing the nitrate-reductase phenotype. These comprised the molypdoterin co-factor biosynthesis genes, flavodoxin protein and nitrate-reductase genes synthesize either structural proteins of the nitrate reductase complex or co-factors and the nitrite-extrusion protein encoding gene (table 4). The other genes are co-conserved in this genetic island among the other strains, and it suggests they have a function in, or associated with, the ability to reduce nitrate. Among these are 7 genes coding for proteins of unknown function, and operon coding for an iron chelating ABC transporter a response regulator and putative sensor protein. The 23 genotyped Lb. plantarum sp. were tested on nitrate-MRS in conditions that shows high nitrite production for Lb. plantarum WCFS1. Of the 23 strains surprisingly only 3 showed the ability to produce nitrite, all of these had the nitrate-reductase island.

The genome of Lactobacillus plantarum WCFS1 additionally shows the presence of the cydABCD operon coding for a cytochrome oxidase: menaquinol-oxidase bd. In Lactococcus lactis MG1363 the functionality of the menaquinol-oxidase bd in an electron transport chain has been recently demonstrated (Brooijmans et. al., 2007). When grown aerobically, supplementation of the growth medium with both heme and menaquinone also gives rise to a respiratory phenotype (tab 5). We therefore propose the existence of an actual branched electron transport chain in Lactobacillus plantarum WCFS1 (FIG. 2).

narG-Mutant and Wild-type Nitrate-Reductase Activities

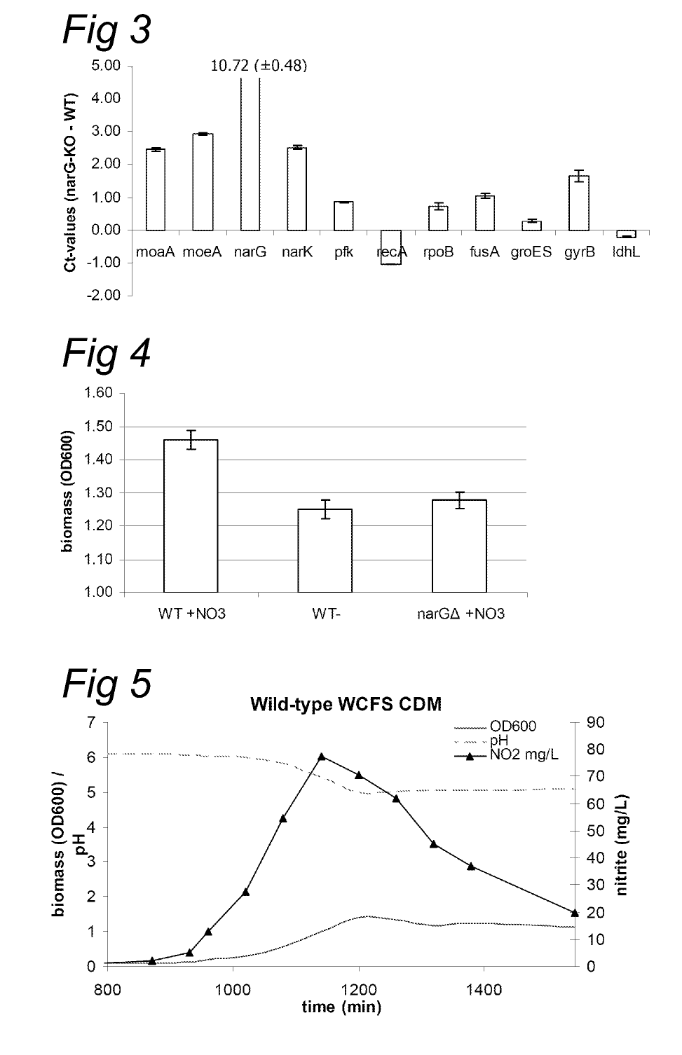

A narG-mutant (narGΔ) was constructed to study involvement of the narGHJI operon in nitrate-reduction. Under the optimal nitrate-inducing conditions for the wild-type narGΔ was unable to produce nitrite. The nitrate-reductase complex requires besides heme also the molybdopterin cofactor. The genome of Lb. plantarum contains all the 9 genes necessary to synthesize molybdopterin and also the nitrite-protein encoding gene narK (10). To investigate the effect of the absence of nitrate reductase activity in the narGΔ strain on the expression levels of the nitrate-reductase, molybdopterin biosynthesis and the nitrite-extrusion protein encoding genes, Q-PCR was performed on wild-type and narGΔ cells (FIG. 3). As mentioned in the introduction, active transcription of the molybdopterin-biosynthesis, nitrate-reductase operon and the nitrite extrusion protein takes place. In the absence of narG mRNA in the narGΔ cells, the transcription levels of the molybdopterin coding genes moeA and moaA, and the nitrite extrusion protein narK were down-regulated, and no transcription of the knock-out narG genes was observed.

Metabolic Impact of Nitrate-Reduction

Nitrate reduction by the nitrate-reductase complex is associated in many organisms with energy-production in two ways. First, energy can be produced via proton motive force generation by the nitrate-reductase complex in the electron transport chain. Second, energy can be generated by the oxidation of non-fermentable substrates, such as L-lactate. Theoretically, as in oxidative respiration, in Lb. plantarum L-lactate formed during fermentation could be taken up and converted to via pyruvate acetate, thereby generating one ATP/L-lactate. Lb. plantarum cells grown overnight under nitrate reducing conditions can oxidize L-lactate to acetate in a phosphate buffer with the concomitant reduction of nitrate into nitrite (tab 6). Cells grown in nitrate-MRS also confirm that a change fermentation patterns takes place (data not shown). When reducing nitrate, more acetate is produced and slightly less L-lactate. Under these conditions a 15% increase in biomass is observed compared to the narGΔ, grown in the same medium, or to wild-type cells grown in the same medium excepting the addition of nitrate (FIG. 4). Since, growth at 10 mM does not lead to pH-inhibited conditions it is safe to say the 15% difference in biomass reflects an actual difference in growth efficiency.

### The Fate of Nitrite

Many Lactobacilli species are known to be able to reduce nitrite to mainly ammonia and NOX (Dodds, K. L., et al, 1985, Appl Environ Microbiol 50:1550-2, Sobko, T., L. et al., 2006. Free Radic Biol Med 14:985-91, and Xu, J. et al, 2001. Appl Microbiol Biotechnol 56:504-7). When the nitrite production of Lb. plantarum is followed during growth in nitrate-CDM, interestingly there is a big build-up of nitrite coinciding with cell-growth, followed by a dramatic decrease of nitrite (FIG. 5). In order to ascertain if nitrite is actively reduced by Lb. plantarum cells and if it shows the same sensitivity to glucose concentration as nitrate-reduction, cells were grown overnight in CDM containing heme, vitamin K2 and nitrite. When compared with medium that has been incubated overnight without cells it is clear that Lb. plantarum actively reduces nitrite. The addition of increasing levels of glucose coincided with higher biomass production but also with increased nitrite reduction (table 6). When nitrate reducing cells are re-suspended in buffer containing L-lactate and nitrate a substantial production of ammonia is also observed. This increase in ammonia is also observed when cells are incubated with only nitrite. Since cells which have been incubated with only L-lactate and no nitrate or nitrite source fail to produce these high levels of ammonia, ammonia is likely derived from nitrite. Since ammonia can be used as a biologically available nitrogen source, nitrate and nitrite can therefore indirectly be used as a nitrogen source.

### Conclusions

For the first time we have found experimental evidence that link the presence of the narGHJI genes to actual nitrate-reduction in lactobacillus plantarum WCFS 1 by construction of the narG mutant (narGΔ). This mutant is unable to reduce nitrate under conditions that allow nitrate reduction by wild-type cells. Further evidence of active transcription of genes involved in nitrate reduction was found by Q-PCR. Q-PCR showed active transcription of molybdopterenin-biosynthesis genes, narK (nitrite extrusion protein encoding gene) and narG, coding for a principal component of the nitrate-reductase complex itself. Q-PCR also verified the absence of narG-mRNA in the narGΔ.

The narGHJI-operon shows a high homology in amino acid sequence to the well-studied E. coli operon, which requires a heme-cofactor to function. The requirement of the heme-cofactor for nitrate-reduction in Lb. plantarum was demonstrated. The E. coli operon additionally requires a molybdopterin cofactor. The presence of molybdopterin biosynthesis genes in the genome and indications for their active transcription in Lb. plantarum was also observed. Besides Lb. plantarum WCFS1 recent draft genomes made available by DOE Joint Genome Institute of Lactobacillus reuteri 100-23 also revealed the presence of a narGHJI operon (http://genome.jgi-psf.org/draft_microbes/lacro/lacro.home.html). Thus the ability to reduce nitrate may not be restricted to the Lb. plantarum strains, but can be found in other lactic acid bacteria. Studies on the nitrate-reductase activities on Lb. plantarum WCFS 1 can be used as a model for nitrate-reductase activities by other LAB.

23 strains of Lb. plantarum were genotyped using WCFS as a comparison. Roughly half of the strains tested showed the presence of the nitrate-reductase genes (narGHJI). In the vicinity of narGHJI, on the chromosome of Lb. plantarum WCFS1, lie genes coding for the biosynthesis of molybdopterin- and the nitrite-extrusion protein (nark). In the 23 genotyped Lb. plantarum strains these genes are either all present or all absent, suggesting a similar nitrate-reductase “island-like” genomic topography in these strains. Interestingly other genes are also present in this 25 kb “nitrate-reductase” island, suggesting their involvement with the reduction of nitrate. Among these genes are various unknown proteins and a iron-chelating ABC-transporter. Only three strains actually showed production of nitrite in culture, these correlated with the presence of the nitrate-reductase island in their genome. Nitrate reductase seems to be widespread among Lb. plantarum strains although the exact conditions for each strain to reduce nitrate to nitrite could differ from species to species.

The requirement of vitamin-K2 addition to the growth-medium for nitrate-reduction reflects that it functions in an electron transport chain system in Lb. plantarum. In E. coli and other organisms proton motive force generation by this type of nitrate-reductase complex has been demonstrated. Under conditions where we observed high nitrite production by Lb. plantarum we indeed see that there is a significant increase in biomass, compared to the NarGΔ-strain or wild-type cells grown in the absence of nitrate. The homologeous nitrate-reductase complex in E. coli is known to function in a electron transport chain context, directly generation proton motive force. The increase in growth biomass demonstrated by nitrate reducing Lb. plantarum cells can be due to formation of PMF by the electron transport chain in Lb. plantarum. Indirectly metabolic energy can also be generated via a shift in redox-balance by an active ETC. We have observed that L-lactate can be converted to acetate, under conditions where nitrate is reduced into nitrite. Conversion of L-lactate via pyruvate to acetate can generate metabolic energy in the form of ATP. Since L-lactate is the main product of anaerobic fermentation by Lb. plantarum growing on glucose also shows higher acetate production concomitant with low L-lactate yield, suggesting this occurs in normal nitrate-reducing culture conditions.

A factor obscuring nitrate-reduction by LAB species is, besides the dependence on co-factors such as heme and a menaquinone-source, the effect of glucose concentrations on nitrogen metabolism. A standard assay method for nitrate-reduction relies mainly on demonstrating the formation of nitrite. At high glucose levels we do not observe a significant production of nitrite. Concomitant with a high production of nitrite we observe a decrease in nitrate levels (FIG. 1). Nitrite production starts almost immediately with the onset of the exponential growth phase (FIG. 5). The lack of nitrate-reductase activity at high glucose levels therefore seems due to catabolic repression rather then a pH-effect.

Reduction of nitrite by Lactobacilli sp. into ammonia, NO and N₂O was known and we have confirmed nitrite reduction by Lb. plantarum WCFS 1 to ammonia which is independent of addition of heme or vitamin-K2 (23-25). As demonstrated by the ability to oxidize the non-fermentable substrate L-lactate, under anaerobic conditions, nitrate-reduction increases the range of usable carbon sources for Lb. plantarum. In addition, nitrate-reduction allows for more efficient growth on fermentable substrates. The clear characterization of the conditions necessary to allow high-levels of nitrate-reduction are therefore of interest for commercial applications of Lb. plantarum. What is especially interesting is the demonstration of the production of high levels of nitrite followed by subsequent removal (FIG. 5). The inhibitory effect of nitrite on growth on various non-desirable species such as clostridae during cheese production is well documented. Addition of heme, vitamin-K2 and nitrate to anaerobic fermentations where Lb. plantarum is used is thus a possible method of naturally preventing outgrowth of non-desired organisms.

TABLE 1
PCR primers used in this study

| PCR-amplify | Forward primer | Reverse primer |
|---|---|---|
| 1 kb upstream narG | P101: CCAGTCAGTAATAGCTGCTAA | P100: CGATAAGACCTCCTTTATCAC |
| 1 kb downstream narG | P102: CGGAAGTTAAAGAAGGTGAAC | P103: CGAATTCTGAGCAGCTTCCA |
| moaA Q-PCR | GCAAAATGATGACGAAGTCCTAGA | TATTCTTTTTGCCAGGTCTTTAATGA |
| moeA Q-PCR | GTCGTCGTGATGCTCGAAAA | TCGGGAACCACGATGTTGAT |
| narG Q-PCR | GTTTGCGGACAACTGGTTAGC | TCTTGCAAAATAACGTGGGTCAT |
| narK Q-PCR | GCCACAAGTAACAGCAGGCTTA | CCCCCAATTGGTCGAACA |
| groES Q-PCR | CCCAAAGCGGTAAGGTTGTT | CTTCACGCTGGGGTCAACTT |
| gyrB QPCR | GGAATTGATGAAGCCCTAGCAG | GAATCCCACGACCGTTATCA |
| IdhL Q-PCR | TGATCCTCGTTCCGTTGATG | CCGATGGTTGCAGTTGAGTAAG |
| pfk Q-PCR | GTGGCGACGGTTCTTACCAT | CCCTGGAAGACCAATCGTGT |
| recA Q-PCR | GGCAGAACAGATCAAGGAAGG | TATCCACTTCGGCACGCTTA |
| rpoB Q-PCR | CACCGTACCCGTAGAAGTTATGC | GGAGACCTTGATCCAAGAACCA |
| fusA Q-PCR | CCCATGATGGTGCTTCACAA | TCGTGGCAGCAGAGGTAATG |
| 16S Q-PCR | TGATCCTGGCTCAGGACGAA | TGCAAGCACCAATCAATACCA |

TABLE 2
Wild-type cells were grown overnight in chemically defined medium
containing 40 mM Nitrate and 10 mM glucose. When both heme and
vitamin K2 are added to the medium a 33-fold increase in nitrite
production is observed.

| | Nitrite mg/L | | |
|---|---|---|---|
| Culture, additions | Average | | stdev |
| wild-type, heme, vit. K2 | 33.54 | ± | 7.11 |
| wild-type, heme | 1.13 | ± | 0.08 |
| wild-type, vit. K2 | 1.31 | ± | 0.04 |
| narGΔ, heme, vit. K2 | Nd | | |

Nd, not detected

TABLE 3
narGHJI genes of Lb. plantarum WCFS1 show high similarity with the
homologeous genes in Lb. reuteri 100-23 and to a lesser extend with
the E. coli genes.

| ORF Lb. | | | |
|---|---|---|---|
| plantarum | Hit to Bacillus | % identical/ | |
| WCFS1 | subtilis 168 | positives^{a} | identity |
| lp_1497 | narG | 55%/72% | nitrate reductase, alpha chain [EC: 1.7.99.4] |
| lp_1498 | narH | 63%/77% | nitrate reductase, beta chain [EC: 1.7.99.4] |
| lp_1499 | narJ | 31%/43% | nitrate reductase, delta chain [EC: 1.7.99.4] |
| lp_1500 | narI | 40%/59% | nitrate reductase, gamma chain [EC: 1.7.99.4] |
| | Hit to Lb. reuteri | | |
| | 100-23 | | |
| lp_1497 | narG | 71%/83% | nitrate reductase, alpha chain [EC: 1.7.99.4] |
| lp_1498 | narH | 86%/93% | nitrate reductase, beta chain [EC: 1.7.99.4] |
| lp_1499 | narJ | 56%/77% | nitrate reductase, delta chain [EC: 1.7.99.4] |
| lp_1500 | narI | 61%/79% | nitrate reductase, gamma chain [EC: 1.7.99.4] |

^{a}Positives are identical AA residues and conserved substitutions

TABLE 4
Fluorescence ratio (respiration vs. aeration) of genes present on the nitrate-reductase
island of Lactobacillus plantarum WCFS1. An up-regulation is observed under
respiratory conditions (when heme and vitamin K2 are present in the medium) of
especially the nitrate-reductase synth. and molybdopterin biosynthesis genes (bold).

| | | | avrg. | stdev. |
|---|---|---|---|---|
| gene | name | product | ratio | probes |
| lp_1473 | fecB | iron chelatin ABC transporter, substr. binding prot. (put.) | 0.92 | 0.16 |
| lp_1475 | fecE | iron chelatin ABC transporter, ATP-binding protein | 1.09 | 0.08 |
| lp_1476 | fecD | iron chelatin ABC transporter, permease protein | 1.18 | 0.08 |
| lp_1477 | lp_1477 | flavodoxin | 1.20 | 0.20 |
| lp_1478 | moaE | molybdopterin biosynthesis protein, E chain | 1.35 | 0.17 |
| lp_1479 | moaD | molybdopterin biosynthesis protein, D chain | 1.47 | 0.21 |
| lp_1480 | moaA | molybdopterin precursor synthase MoaA | 1.36 | 0.14 |
| lp_1481 | narK | nitrite extrusion protein | 1.56 | 0.24 |
| lp_1483 | lp_1483 | unknown | 1.10 | 0.09 |
| lp_1484 | lp_1484 | unknown | 1.26 | 0.13 |
| lp_1485 | lp_1485 | unknown | 1.50 | 0.17 |
| lp_1486 | lp_1486 | unknown | 1.54 | 0.28 |
| lp_1487 | rrp4 | response regulator | 1.02 | 0.09 |
| lp_1488 | hpk4 | histidine protein kinase; sensor protein (putative) | 1.08 | 0.10 |
| lp_1489 | lp_1489 | unknown | 1.12 | 0.06 |
| lp_1490 | lp_1490 | unknown | 1.23 | 0.17 |
| lp_1491 | mobA | molybdopterin-GD biosynth. prot. MobA (putative) | 1.19 | 0.11 |
| lp_1492 | moaC | molybdopterin precursor synthase MoaC | 1.32 | 0.12 |
| lp_1493 | mobB | molybdopterin-GD biosynthesis protein MobB | 1.34 | 0.11 |
| lp_1494 | moeA | molybdopterin biosynthesis protein MoeA | 1.40 | 0.25 |
| lp_1495 | moaB | molybdopterin biosynthesis protein MoaB | 1.44 | 0.10 |
| lp_1496 | moeB | molybdopterin biosynthesis protein MoeB | 1.33 | 0.25 |
| lp_1497 | narG | nitrate reductase, alpha chain | 1.58 | 0.18 |
| lp_1498 | narH | nitrate reductase, beta chain | 1.67 | 0.26 |
| lp_1499 | narJ | nitrate reductase, delta chain | 1.49 | 0.11 |
| lp_1500 | narI | nitrate reductase, gamma chain | 1.53 | 0.26 |
| lp_1502 | lp_1502 | unknown | 1.29 | 0.28 |
| lp_1503 | lp_1503 | unknown | 1.16 | 0.18 |

TABLE 5
1 Respiratory-like phenotype displayed by Lactobacillus plantarum
WCFS1 when grown in the presence of both heme and a quinine
source (vitamin K2)

| | | |
|---|---|---|
| Lb. plantarum | | |
| WCFS1 | Biomass | Acidity |

| Heme | Vitamin K2 | (OD₆₀₀) | (pH) |
|---|---|---|---|
| + | + | 9.45 (±0.16) | 4.39 (±0.06) |
| + | − | 5.45 (±0.07) | 3.94 (±0.01) |
| − | + | 4.71 (±0.21) | 3.94 (±0.01) |

TABLE 6
Cells were washed in 50 mM potassium phosphate buffer (pH 5.0), re-suspended
to OD₆₀₀ of 2.0, in buffer containing 10 mM L-lactate and, where indicated,
20 mM nitrate or 20 mM nitrite was added. After overnight incubations ammonia,
L-lactate, acetate and nitrite concentrations were measured. The cells were
wild-type or narGΔ, pre-cultured on nitrate-MRS.

| | | Nitrite | ammonia | L-lactate (mM) | Acetate |
|---|---|---|---|---|---|
| Strain | Addition | (mM) | (uM) | consumed | (mM) |
| wild-type | NO₃ | 4.36 (±0.18)^{a} | 1002 (±27) | 7.49 (±0.22) | 4.51 (±0.26) |
| narGΔ | NO₃ | Nd | 44 (±0.67) | 3.67 (±2.21) | 0.96 (±0.51) |
| wild-type | — | Nd | 50 (±1.35) | 1.03 (±0.52) | 1.02 (±0.08) |
| wild-type | NO₂ | 1.72 (±1.23)^{b} | 448 (±89) | 2.96 (±0.58) | 1.56 (±0.12) |
| — | NO₃ | Nd | Nd | Nd | Nd |

Nd, not detected
^{a}nitrite, produced
^{b}nitrite, consumed

APPENDIX 1
Composition of chemically defined medium (CDM)

| CDM component | Concentration (g/liter) |
|---|---|
| Vitamins | |
| Ca-(D)-(+)-pantothenate (vitamin B5) | 0.001 |
| D-Biotin (vitamin B7)c | 0.0025 |
| Folic acid (vitamin B11) | 0.001 |
| Lipoic acid (6,8-thioctic acid) | 0.001 |
| Nicotinic acidc | 0.001 |
| D-Aminobenzoic acid | 0.01 |
| Pyridoxamine HCl | 0.005 |
| Pyridoxine HCl (vitamin B6) | 0.002 |
| Pyridox-xc | |
| Riboflavin (vitamin B2) | 0.001 |
| Thiamine HCl (vitamin B1) | 0.001 |
| Amino acids | |
| Alanine | 0.24 |
| Arginine | 0.125 |
| Aspartic acid | 0.42 |
| Cysteine-HCl | 0.13 |
| Glutamic acid | 0.5 |
| Glycine | 0.175 |
| Histidine | 0.15 |
| Isoleucine | 0.21 |
| Leucine | 0.475 |
| Lysine | 0.44 |
| Methionine | 0.125 |
| Phenylalanine | 0.275 |
| Proline | 0.675 |
| Serine | 0.34 |
| Threonine | 0.225 |
| Tryptophan | 0.05 |
| Tyrosine | 0.25 |
| Valine | 0.325 |
| Nucleotides | |
| Adenine | 0.01 |
| Guanine | 0.01 |
| Inosine | 0.005 |
| Orotic acid (vitamin B13) | 0.005 |
| Thymidine | 0.005 |
| Uracil | 0.01 |
| Xanthine | 0.01 |
| Additional components | |
| K2HPO4 | 1.0 |
| KH2PO4 | 5.0 |
| sodium acetate | 1.0 |
| ammonium citrate | 0.6 |
| ascorbic acid (vitamin C) | 0.5 |

### REFERENCE LIST

1. Ahrne, S., S, Nobaek, B. Jeppsson, I. Adlerberth, A. E. Wold, and G. Molin. 1998. The normal Lactobacillus flora of healthy human rectal and oral mucosa. J Appl Microbiol 85:88-94.
2. Arneth, W., and B. Herold. 1988. Nitrat/Nitrit-Bestimmung in Wurstwaren nach enzymatischer Reduction. Fleischwirtschaft 68:761-764.
3. Bergmeyer, H. U. 1974. Methods of Enzymatic Analysis, 2nd. ed, vol. 1. Weinheim/Academic Press Inc., New York and London.
4. Bergmeyer, H. U., and H. Möllering. 1974. Methods of Enzymatic Analysis, 2nd. ed, vol. 3. Weinheim/Academic Press Inc., New York and London.
5. Beutler, H.-O. 1984. Methods of Enzymatic Analysis, 3rd ed. ed, vol. VI. Deerfield Beach/Florida, Basel.
6. Beutler, H.-O., B. Wurst, and S. Fisher. 1986. Eine neue Methode zur enzymatischen Bestimmung van Nitrat in Lebensmitteln. Deutsche Lebensmittel-Rundschau 82:283-289.
7. Costilow, R. N., and T. W. Humphreys. 1955. Nitrate reduction by certain strains of Lactobacillus plantarum. Science 121:168.
8. Gangolli, S. D., P. A. van den Brandt, V. J. Feron, C. Janzowsky, J. H. Koeman, G. J. Speijers, B. Spiegelhalder, R. Walker, and J. S. Wisnok. 1994. Nitrate, nitrite and N-nitroso compounds. Eur J Pharmacol 292:1-38.
9. Gutmann, I., and A. W. Wahlefeld. 1974. Methoden der enzymatischen Analyse, 2nd ed. ed, vol. 3. Weinheim/Academic Press Inc., New York and London.
10. Kleerebezem, M., J. Boekhorst, R. van Kranenburg, D. Molenaar, O. P. Kuipers, R. Leer, R. Tarchini, S. A. Peters, H. M. Sandbrink, M. W. Fiers, W. Stiekema, R. M. Lankhorst, P. A. Bron, S. M. Hoffer, M. N. Groot, R. Kerkhoven, M. de Vries, B. Ursing, W. M. de Vos, and R. J. Siezen. 2003. Complete genome sequence of Lactobacillus plantarum WCFS1. Proc Natl Acad Sci USA 100:1990-5.
11. Kretzschmar, R., and T. Kretschmar. 1988. Enzymatische Nitrat-Bestimmung in kommunalen Abwasser. Vom Abwasser 70:119-128.
12. Linseisen, J., S. Rohrmann, T. Norat, C. A. Gonzalez, M. Dorronsoro Iraeta, P. Morote Gomez, M. D. Chirlaque, B. G. Pozo, E. Ardanaz, I. Mattisson, U. Pettersson, R. Palmqvist, B. Van Guelpen, S. A. Bingham, A. McTaggart, E. A. Spencer, K. Overvad, A. Tjonneland, C. Stripp, F. Clavel-Chapelon, E. Kesse, H. Boeing, K. Klipstein-Grobusch, A. Trichopoulou, E. Vasilopoulou, G. Bellos, V. Pala, G. Masala, R. Tumino, C. Sacerdote, M. Del Pezzo, H. B. Bueno-de-Mesquita, M. C. Ocke, P. H.

Peeters, D. Engeset, G. Skeie, N. Slimani, and E. Riboli. 2006. Dietary intake of different types and characteristics of processed meat which might be associated with cancer risk—results from the 24-hour diet recalls in the European Prospective Investigation into Cancer and Nutrition (EPIC). Public Health Nutr 9:449-64.
13. McKnight, G. M., C. W. Duncan, C. Leifert, and M. H. Golden. 1999. Dietary nitrate in man: friend or foe? Br J Nutr 81:349-58.
14. Mensing a, T. T., G. J. Speijers, and J. Meulenbelt. 2003. Health implications of exposure to environmental nitrogenous compounds. Toxicol Rev 22:41-51.
15. Molenaar, D., F. Bringel, F. H. Schuren, W. M. de Vos, R. J. Siezen, and M. Kleerebezem. 2005. Exploring Lactobacillus plantarum genome diversity by using microarrays. J Bacteriol 187:6119-27.
16. Noll, F. 1966. Methoden zur quantitativen Bestimmung von L(+)-Lactat mittels Lactat-Dehydrogenase and Glutamat-Pyruvat-Transaminase. Biochem Z 346:41-49.
17. Piendl, A., and I. Wagner. 1983. Physiologischen Eigenschaften der organischen Sauren des Bieres. Brauindustrie 68:1520-1528.
18. Rogosa, M. 1961. Experimental conditions for nitrate reduction by certain strains of the genus Lactobacillus. J Gen Microbiol 24:401-8.
19. Sambrook, J., E. F. Fritsch, and T. Maniatis. 1989. Molecular cloning: a laboratory manual, 2nd ed.
20. Stewart, V. 1988. Nitrate respiration in relation to facultative metabolism in enterobacteria. Microbiol. Rev 52:190-232.
21. Vesa, T., P. Pochart, and P. Marteau. 2000. Pharmacokinetics of Lactobacillus plantarum NCIMB 8826, Lactobacillus fermentum KLD, and Lactococcus lactis MG 1363 in the human gastrointestinal tract. Aliment Pharmacol Ther 14:823-8.
22. Wang, H., C. P. Tseng, and R. P. Gunsalus. 1999. The napF and narG nitrate reductase operons in Escherichia coli are differentially expressed in response to submicromolar concentrations of nitrate but not nitrite. J Bacteriol 181:5303-8.
23. Wolf, G., E. K. Arendt, U. Pfahler, and W. P. Hammes. 1990. Heme-dependent and heme-independent nitrite reduction by lactic acid bacteria results in different N-containing products. Int J Food Microbiol 10:323-9.
24. Wolf, G., and W. P. Hammes. 1988. Effect of hematin on the activities of nitrite reductase and catalase in lactobacilli. Arch Microbiol 149:220-224.
25. Xu, J., and W. Verstraete. 2001. Evaluation of nitric oxide production by lactobacilli. Appl Microbiol Biotechnol 56:504-7.
26. Zumft, W. G. 1997. Cell biology and molecular basis of denitrification. Microbiol Mol Biol Rev 61:533-616.

1. Method for reducing nitrate into nitrite wherein a probiotic and/or a starter bacterium is cultivated under anaerobic conditions in the presence of a nitrate, a heme and optionally a vitamin K.

2. A method according to claim 1, wherein no other microorganisms are added.

3. A method according to claim 1, wherein the probiotic and/or starter bacterium has not been genetically modified.

4. A method according to claim 1, wherein the probiotic and/or starter bacterium has been genetically modified to produce a heme without the need for heme addition.

5. A method according to claim 1, wherein the cultivation is carried out in a product, preferably a food product.

6. A method according to claim 1, wherein the formation of nitrite prevents outgrowth of spoilage microorganisms.

7. A method according to claim 1, wherein the probiotic and/or starter bacterium obtained has improved characteristics as to its biomass production and/or its ability to survive in the human or animal gastrointestinal tract.

8. A method according to claim 1, wherein the probiotic and/or starter bacterium is a lactic acid bacteria or a Bifidobacteria.

9. A method according to claim 8, wherein the lactic acid bacteria is selected among the following species: Lactobacillus, Streptococcus, Lactococcus Pediococcus or Leuconostoc.

10. A method according to claim 1, wherein a heme is present in an amount which is ranged between 1.25 and 50 μg/ml and optionally a vitamin K between 5 and 100 μg/ml.
